(19) United States
(12) Patent Application Publication
(10) Pub. No.: US 2025/0035554 A1
(43) Pub. Date: Jan. 30, 2025

(54) SYSTEM AND METHOD FOR SPECTROSCOPIC DETERMINATION OF A CHEMOMETRIC MODEL FROM SAMPLE SCANS

(71) Applicant: THERMO SCIENTIFIC PORTABLE ANALYTICAL INSTRUMENTS INC., Tewksbury, MA (US)

(72) Inventors: Nikita Khainovski, Marlborough, MA (US); Lin ZHANG, Andover, MA (US); Nimesh Khadka, Dracut, MA (US)

(21) Appl. No.: 18/782,763

(22) Filed: Jul. 24, 2024

Related U.S. Application Data
(60) Provisional application No. 63/515,638, filed on Jul. 26, 2023.

(51) Int. Cl. G01N 21/65; G06F 30/20; G16C 20/70

(52) U.S. Cl. CPC G01N 21/65; G06F 30/20; G16C 20/70

(57) ABSTRACT

A computer-implemented method and system are provided. An analytical instrument support system receives a first spectra dataset associated with scans of one or more first samples, the one or more first samples including a target analyte having one or more known levels of a parameter. The analytical instrument support system receives a second spectra dataset associated with scans of one or more second samples. The analytical instrument support system determines one or more spectra arrays by combining (i) the first spectra dataset and (ii) the second spectra dataset. The analytical instrument support system determines a chemometric model for one or more levels of the parameter of the target analyte based on, at least, the one or more spectra arrays and the one or more known levels of the parameter.

### RELATED APPLICATIONS

This application claims priority to U.S. Provisional Patent Application No. 63/515,638 filed Jul. 26, 2023, the entire content of which is incorporated by reference herein.

### FIELD

The present disclosure generally relates to systems and methods for conducting spectroscopic analytical techniques, such as Raman spectroscopy. In particular, systems and methods are disclosed for determining a chemometric model for an unknown parameter of an analyte based on one or more spectra simulations and one or more known levels of a parameter.

### BACKGROUND

Raman spectroscopy is an effective tool for identifying and characterizing various sample compounds and substances. In Raman spectroscopy, light typically from a laser and of a known wavelength (typically infrared or near infrared) is directed at a sample compound or substance. The laser light (also sometimes referred to as a Raman pump) interacts with the electron clouds in the molecules of the sample compound or substance and, as a result of this interaction, experiences selected wavelength shifting. The precise nature of this wavelength shifting depends upon the materials present in the sample compound or substance. A unique wavelength signature (typically called the Raman signature) is produced by each sample compound or substance. This unique Raman signature permits the sample compound or substance to be identified and characterized. More specifically, the spectrum of light returning from the sample compound or substance is analyzed with a spectrometer so as to identify the Raman-induced wavelength shifting in the Raman pump light, and then this wavelength signature is compared (e.g., by a computing device) with a library of known Raman signatures, whereby to identify the precise nature of the sample compound or substance.

Raman spectroscopy has scientific, commercial and public safety applications. Methods and systems for sampling analytes and matrices often include more than 5 distinct reactor runs with each run taking an average of 15 days to complete. Those distinct reactor runs can be used as training datasets to build a chemometric model but take significant resources over long periods of time (e.g., 6 months or more) to complete.

### SUMMARY

In one aspect, a computer-implemented method is disclosed. One or more processors receive a first spectra dataset associated with scans of one or more first samples, the one or more first samples including a target analyte having one or more known levels of a parameter. One or more processors receive a second spectra dataset associated with scans of one or more second samples. One or more processors determine one or more spectra arrays by combining (i) the first spectra dataset and (ii) the second spectra dataset. One or more processors determine a chemometric model for one or more levels of the parameter of the target analyte based on, at least, the one or more spectra arrays and the one or more known levels of the parameter.

In another aspect, an analytical instrument support system is disclosed. The analytical instrument support system includes one or more computer processors, one or more non-transitory computer-readable storage media, and program instructions stored on at least one of the one or more non-transitory computer readable storage media for execution by at least one of the one or more processors. The program instructions include program instructions to (i) receive a first spectra dataset associated with scans of one or more first samples, the one or more first samples including a target analyte having one or more known levels of a parameter; (ii) receive a second spectra dataset associated with scans of one or more second samples; (iii) determine one or more spectra arrays by combining (i) the first spectra dataset and (ii) the second spectra dataset; and (iv) determine a chemometric model for one or more levels of the parameter of the target analyte based on, at least, the one or more spectra arrays and the one or more known levels of the parameter.

In another aspect, an analytical instrument is disclosed. The analytical instrument includes a light source configured to direct light onto a surface of a sample and a spectrograph to acquire a Raman spectrum from the surface of the sample in response to the light source directing light onto the surface of the sample. The analytical instrument further includes one or more computer processors, one or more non-transitory computer-readable storage media. Upon execution of the program instructions by at least one of the one or more processors, the analytical instrument is caused to implement the following acts, including (i) receive a first spectra dataset associated with scans of one or more first samples, the one or more first samples including a target analyte having one or more known levels of a parameter; (ii) receive a second spectra dataset associated with scans of one or more second samples; (iii) determine one or more spectra arrays by combining (i) the first spectra dataset and (ii) the second spectra dataset; and (iv) determine a chemometric model for one or more levels of the parameter of the target analyte based on, at least, the one or more spectra arrays and the one or more known levels of the parameter.

There is no specific requirement that a system, method, or technique relating to determination-based spectroscopy include all of the details characterized herein, in order to obtain some benefit according to the present disclosure. Thus, the specific examples characterized herein are meant to be exemplary applications of the techniques described, and alternatives are possible.

### BRIEF DESCRIPTION OF THE DRAWINGS

Features and advantages of the present technology will become more apparent from the following detailed description of exemplary embodiments thereof taken in conjunction with the accompanying drawings in which:

FIG. 1 is a block diagram of an exemplary analysis system, according to some implementations of the present disclosure.

FIG. 2 illustrates an optical architecture for a spectrometer included in the analysis system of FIG. 1, according to some implementations of the present disclosure.

FIG. 3 illustrates another optical architecture for the spectrometer included in the analysis system of FIG. 1, according to some implementations of the present disclosure.

FIG. 4 illustrates a further optical architecture for the spectrometer included in the analysis system of FIG. 1, according to some implementations of the present disclosure.

FIG. 5 illustrates yet another optical architecture for the spectrometer included in the analysis system of FIG. 1, according to some implementations of the present disclosure.

FIG. 6 illustrates an exemplary reactor, according to some implementations of the present disclosure.

FIG. 7 is a flowchart of an exemplary process to determine a chemometric model for an unknown parameter of an analyte, according to some implementations of the present disclosure.

FIG. 8A illustrates exemplary spectra of one or more analytes, according to some implementations of the present disclosure.

FIG. 8B illustrates exemplary spectra of one or more matrices, according to some implementations of the present disclosure.

FIG. 9 illustrates the determination of an exemplary chemometric model, according to some implementations of the present disclosure.

FIG. 10 illustrates the determination of another exemplary chemometric model, according to some implementations of the present disclosure.

FIG. 11A illustrates the determination of the exemplary chemometric model for predicting an analyte concentration level based on, at least, a first reactor, according to some implementation of the present disclosure.

FIG. 11B illustrates the determination of the exemplary chemometric model for predicting an analyte concentration level based on, at least, a second reactor, according to some implementations of the present disclosure.

FIG. 11C illustrates the determination of the exemplary chemometric model for predicting an analyte concentration level based on, at least, a third reactor, according to some implementations of the present disclosure.

FIG. 11D illustrates the determination of the exemplary chemometric model for predicting an analyte concentration level based on, at least, a fourth reactor, according to some implementations of the present disclosure.

FIG. 11E illustrates the determination of the exemplary chemometric model for predicting an analyte concentration level based on, at least, a fifth reactor, according to some implementations of the present disclosure.

FIG. 12A illustrates concentrations of lactate vs glucose, according to some implementations of the present disclosure.

FIG. 12B illustrates concentrations of glutamine vs lactate, according to some implementations of the present disclosure.

FIG. 12C illustrates concentrations of glutamine vs glucose, according to some implementations of the present disclosure.

While the present technology is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

Systems, methods and techniques are disclosed herein for chemometric modeling that is applied to determine an unknown parameter of an analyte based on spectroscopic scans of one or more samples, such as samples of physical compounds or biological substances. The present disclosure can be particularly desirable by providing chemometric model determinations that are rapidly obtainable relative to current state-of-the-art methods and systems. For example, the present disclosure provides improved methods for determining one or more spectra simulations (arrays) based on, at least, a first spectra dataset associated with one or more reactors collected from scans of one or more first samples and a second spectra dataset associated with one or more reactors collected from scans of one or more second samples. The present disclosure desirably provides improved methods by determining the chemometric model without performing a large number of reactor runs to collect the first spectra dataset and the second spectra dataset, thereby improving efficiency by reducing the amount resources used during current-state-of-the art reactor runs. The improvements of the present disclosure include rapid and inexpensive determination of chemometric models for the unknown parameter of the analyte based on, at least, one or more determined spectra simulations and one or more known levels of the parameter of the analyte. The present disclosure also desirably provides improved methods for determining chemometric models without comprising the accuracy of the acquisition of the unknown parameter of the analyte. The improvements of the present disclosure include high signal-to-noise ratio (SNR) for the determination of chemometric models for the unknown parameter, thereby improving the sensitivity and specificity compared against current-state-of-the-art reactor runs which include poor signal saturation.

As described herein, the Raman measurement parameters for the analytic system are initial targets provided as instructions to the analytic instrument for obtaining a spectrum. The Raman measurement parameters can include scan time and one or more Raman shift wavenumbers. The system and methods described herein model these Raman measurement parameters to determine a chemometric model for a parameter of an analyte. For example, a first spectra dataset is collected from Raman spectroscopy scans of one or more first samples of an analyte, where the analyte has known levels of a parameter and a second spectra dataset is collected from Raman spectroscopy scans of one or more second samples, where the second samples include a matrix. A chemometric model is determined for an unknown parameter of the analyte based on, at least, the first spectra dataset and the second spectra dataset.

Unless otherwise defined, all technical and scientific terms used herein have the same meaning as commonly understood by one of ordinary skill in the art. In case of conflict, the present document, including definitions, will control. Example methods and systems are described below, although methods and systems similar or equivalent to those described herein can be used in practice or testing of the present disclosure. All publications, patent applications, patents and other references mentioned herein are incorporated by reference in their entirety. The systems, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A, X employs B, or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form.

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Raman measurement” refers to a Raman system where the illumination spot diameter remains fixed-size and has a uniform radial distribution.

“Aspheric diffuse ring producing optic” refers to various implementations for producing the distributed spot which includes an aspheric diffuse ring producing optic, or ADRPO. In some implementations, aspheric optics may include what is referred to as an axicon or conical optic which produces a ring of intensity but has higher order aspheric terms to produce the spread-out pattern. In some implementations, the aspheric optic may have coefficients of Al=0.01, A2=0.06, and A4=0.002, with all other terms being zero.

“Collimating lens” refers to optical elements that transform the incoming light direction to parallel paths.

“Filter” refers to optical elements that remove some wavelengths of incoming light.

“Focusing optics” refers to optical elements that transform the incoming light direction to a point in space.

“Light source” refers to a light source used for excitation in spectroscopy application. Exemplary systems and methods may include a laser that is adapted for Raman spectroscopy such as 785 m, or 1064 nm. Exemplary light sources could also include a broad band source such as an LED.

“Sample surface plane” refers to the surface of the sample under test where the illumination area is directed.

“Steering mirrors” refers to optical elements used to change the direction of light path.

“Raman spectrum” refers to a spectrum of data values that may include a bright spectrum and/or a dark spectrum. Where the bright spectrum is the scattered light from the sample hitting a detector. The dark spectrum is a spectrum received when no light hits the detector. The dark spectrum captures the shape of the baseline offset.

“Reactor” refers to a manufactured device or system that supports a biologically active or chemically active environment in which a chemical or biochemical reaction is carried out. In some implementations, the reactor is a bioreactor. In some implementations, the reactor is a chemical reactor.

The present disclosure is now described with reference to the drawings, wherein like reference numerals are used to refer to like elements throughout. In the following description, for purposes of explanation, numbers of specific details are set forth in order to provide an improved understanding of the present disclosure. It may be evident, however, that the systems and methods of the present disclosure may be practiced without one or more of these specific details. In other instances, well-known structures and devices are shown in block diagram form in order to facilitate describing the systems and methods of the present disclosure.

It should be understood that although implementations are described herein as being used with a spectrometer or other optical instrument, implementations can be constructed as stand-alone devices for measuring an electrochemical property of a sample compound or substance. Furthermore, although some implementations are described herein with respect to measuring an electrochemical property of a sample compound or substance, exemplary methods and systems described herein can be used to measure other electrochemical properties, such as, for example a Raman spectrum of the sample compound or substance.

Exemplary analysis systems, such as Raman spectroscopy systems, can be used in a variety of environments to identify unknown materials, to evaluate the threat posed by unknown materials, to provide positive identification of packaged raw materials, or to provide general security screening functions of a variety of substances. Exemplary analysis systems can include a wide range of sizes, from portable, handheld instruments to larger systems in permanent laboratories.

### I. Exemplary Analysis Systems

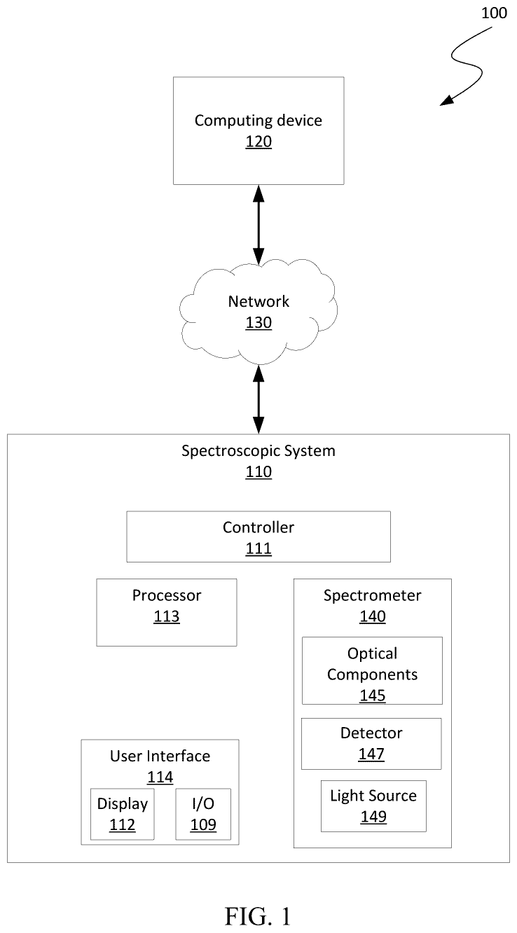

Those of ordinary skill in the art appreciate that there are a variety of different optical architectures and arrangements utilized in the field of Raman spectroscopy. FIG. 1 provides an illustrative example of an analysis system 100 (also referred to herein as “analyzer 100”) that comprises an optical architecture and other elements that operate to measure one or more Raman spectra from a sample via one or more of the methods described herein.

The analyzer 100 illustrated in FIG. 1 includes a spectroscopic system 110 communicatively coupled to a computing device 120 via a network 130. As illustrated in FIG. 1, the spectroscopic system 110 includes a controller 111, an electronic signal processor 113, and a spectrometer 140 (e.g., a Raman spectrometer).

In some implementations, the analyzer 100 provides a stand-alone or dedicated analytical instrument or device (or set of instruments or devices) configured to perform a short scan and analysis of a sample. However, in other implementations, the analyzer 100 may be configured to perform additional scans or analysis of one or more samples. Combining such scanning abilities or analysis in one system (e.g., one analytical instrument) creates desirable efficiencies and improved accuracy in the analysis as multiple scans can be taken from the sample without having to change the position of the sample, reconfigure the analytical instrument, or use a separate scan for additional samples combined with the target sample, all of which can introduce delays and potentials for contamination or unintended variances between scans.

It will be appreciated that, in some implementations, at least a portion of the computing device 120 may be located separate from the spectroscopic system 110, which provides the opportunity for increased computing power at a central location or across multiple locations. One skilled in the art can envision various interconnections, both physical and wireless, between the components of the analyzer 100. It will further be appreciated that, in some implementations, the spectroscopic system 110 and the computing device 120 may be communicatively coupled without the network 130 (e.g., via a dedicated wired or wireless connection). Alternatively, some implementations of the analyzer 100 may not require the resources of computing device 120 but may instead utilize resources internal to the spectroscopic system 110 to perform the methods described herein. Thus, computing device 120 may not be necessary for operation of the analyzer 100 and/or the spectroscopic system 110 and the example of FIG. 1 should not be considered as limiting. As described herein, the analyzer 100 may be used to measure one or more Raman spectra from a sample compound or substances via one or more of the methods described herein.

It should be understood that, in some implementations, the components of the analyzer 100 and/or the spectroscopic system 110 illustrated in FIG. 1 may be included in a common housing forming an analytical instrument that may include a benchtop or a portable Raman spectrometer device (e.g., a handheld device). However, in other implementations, one or more components of the analyzer 100 and/or the spectroscopic system 110 may be contained in separate housings or devices and may be coupled (e.g., communicatively, electrically, mechanically, or the like) as needed to carry out the methods described herein. Also, in some implementations, the operations described herein as being performed by the components of the analyzer 100 and/or the spectroscopic system 110 may be combined and distributed in various ways. For example, in some implementations, the electronic signal processor 113 may be part of the controller 111, wherein the controller 111 is configured to perform the operations of the electrical signal processor 113 as described herein. Furthermore, the operations described herein as being performed by the controller 111 may be distributed among multiple controllers. In the same or alternative examples, operations described herein as being performed by controller 111 may be distributed among one or more computing devices (e.g., the electronic signal processor 113, the computing device 120, or multiple computing devices). In some implementations, the controller 111 is configured to control operation of the spectrometer 140, wherein the electronic signal processor 113 is configured to control other components of the spectroscopic system 110 (e.g., communication with the computing device 120). However, these roles of the controller 111 and the electronic signal processor 113 may be combined and distributed in various ways, and, in some implementations, the spectroscopic system 110 includes only the controller 111 or the electronic signal processor 113 and the included devices performed the functionality of both the controller 111 and the electronic signal processor 113 as described herein.

The spectroscopic system 110 may also include additional components (such as power components), a user interface 114 (such as a display 112 and/or user input and/or output (“I/O”) interface 109, such as, for example, a keyboard, a mouse, a touch screen), optical components (e.g., mirrors, lens, fiber optic cables, gratings, and filters), and the like. The spectrometer 140 included in the spectroscopic system 110 includes one or more optical components 145, a detector 147 (e.g., a CCD detector, a PMT detector, or other detector known in the art), and a light source 149. The light source 149 provides an excitation beam (e.g., excitation laser providing 785 nm or 1064 nm light) to a sample (not shown in FIG. 1).

As described above, the spectroscopic system 110 and/or the spectrometer 140 may comprises a fully integrated portable system operated by a user on battery power to take Raman spectroscopy measurements in a variety of environments, such as, for example, a laboratory setting, a manufacturing (e.g., bioreactor based) setting, a remote setting, etc. Also, in the same or alternative implementations, elements of the spectroscopic system 110 may be utilized as separated systems communicatively connected (e.g., optically, wirelessly, electrically, mechanical, and the like) and operated on battery power and/or power outlets connected to a central power source to take Raman spectroscopy measurements in the variety of environments described.

Referring now to the light source 149 of spectrometer 140, it will be appreciated that implementations of the light source 149 may emit wavelengths of light as needed for an application, for example, including or between a range of about 400 nm to about 1064 nm, a range of about 400 nm to about 750 nm, a range of about 400 nm to about 600 nm, a range of about 400 nm to about 500 nm, a range of about 600 nm to about 900 nm, a range of about 700 nm to about 850 nm, a range of 600 nm to 1064 nm, a range of 750 nm to 1064 nm, a range of 850 nm to 1064 nm, a range of 950 nm to 1064 nm, as well as a wavelength of about 785 nm, or a wavelength of about 1064 nm.

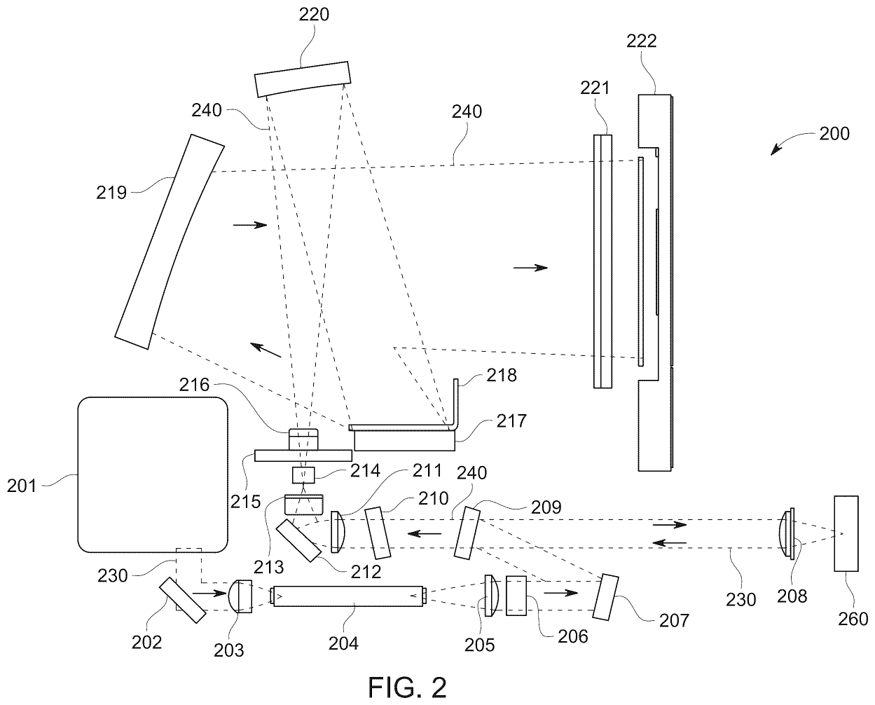

FIG. 2 provides an illustrative example of one implementation of an optical architecture comprising optical components of the spectrometer 140 (see FIG. 1), that are otherwise collectively referred to herein as an optical system 200. It will be appreciated that different optical architectures of Raman spectrometer are known in the art and thus the example of FIG. 2 should not be considered as limiting. For example, some implementations employ what are referred to as transmission gratings rather the reflection gratings, as well as associated differences in optical architecture.

The example of FIG. 2 illustrates one implementation of the light source 149 (see FIG. 1) as a laser assembly 201 comprising a laser source that produces a beam of light that travels along optical or beam path 230 (e.g., arrows illustrate direction of travel of the light beam) to sample 260. It will be appreciated that sample 260 may include any type of sample of interest to a user and may include substantially dry samples (e.g., a powder, solid material), substantially fluid samples (e.g., a liquid, gas), or some combination thereof (e.g., a gel). In response to the light from laser assembly 201, the sample 260 produces scattered light (e.g., comprising a Raman portion and a Rayleigh portion of scattered light), which travels along optical or beam path 240.

In some implementations, the laser assembly 201 may produce laser power as needed for an application for example, including or between a range of about 250 mW to about 750 mW; about 250 mW to about 700 mW; about 250 mW to about 650 mW; about 250 mW to about 600 mW; about 250 mW to about 550 mW; about 250 mW to about 500 mW; about 250 mW to about 450 mW; about 250 mW to about 400 mW; about 250 mW to about 350 mW; about 250 mW to about 300 mW; or about 250 mW. Also in some implementations, the laser power affects the values of the base value and the bright-max intensity values when sample 260 is scanned. It will be appreciated that other ranges and/or levels of laser power are known in the art and thus the example described for laser assembly 201 should not be considered as limiting.

FIG. 2 also illustrates one implementation of an architecture that directionally controls the beam path 230 and the beam path 240 as well as conditions one or more characteristics of the beam of light produced from the laser assembly 201 as well as from the sample 260. For example, a turning mirror 202 redirects beam path 230 to focusing lens 203 that focuses the beam onto a waveguide phase scrambler 204 (e.g., to adjust the phase characteristics of the beam). The beam exits waveguide phase scrambler 204 and travels to a collimating lens 205 (e.g., which adjusts collimation characteristics of the beam), then to a broadband filter 206 transmissive to a specific wavelength or range of wavelengths of light. The beam travels to a flat mirror 207 that redirects the beam path 230 to a selective element 209. It will be appreciated that the selective element 209 may include a dichroic mirror, a notch filter, or other element that comprises substantially reflective characteristics to the wavelength(s) of the beam from laser assembly 201 and comprises substantially transmissive characteristics to a wavelength or wavelength range associated with Raman scattered light from sample 260. In the described example, selective element 209 redirects the beam path 230 to a lens 208 that focuses the beam to the sample 260. In the described example, the lens 208 may include any type of lens known in the art such as an objective lens that focuses the beam onto the sample 260. Also, some implementations of the lens 208 comprise special configurations and characteristics that provide advantages for different types of the sample 260 as will be described below.

The lens 208 collects Raman scattered light and Rayleigh scattered light produced from the sample 260 in response to the beam from the laser assembly 201 and produces the beam path 240 that travels back to the selective element 209 and a second selective element 210. As described above, the selective elements 209 and 210 are substantially transmissive to the wavelengths of the Raman scattered light, allowing the beam path 240 to pass through to additional optical elements that further adjust the path and conditions the characteristics of the beam traveling along the beam path 240. For example, the optical elements may include a focusing lens 211, a flat mirror 212, a baffle 213, a slit 214, a baffle 215, and a collimating lens 216.

The beam path 240 travels from the collimating lens 216 to a mirror 220 that reflects the beam path 240 toward a diffraction grating 217. It will be appreciated that, in the example of FIG. 2, the diffraction grating 217 comprises a reflective diffraction grating that produces a spectral distribution of light. The beam path 240 then travels to a focusing mirror 219 that redirects the beam path 240 to a focusing lens 221 that directs the beam to elements of a detector 222 (one implementation of the detector 147 of FIG. 1). It will also be appreciated that FIG. 2 illustrates a baffle 218 that, in some implementations, controls stray light.

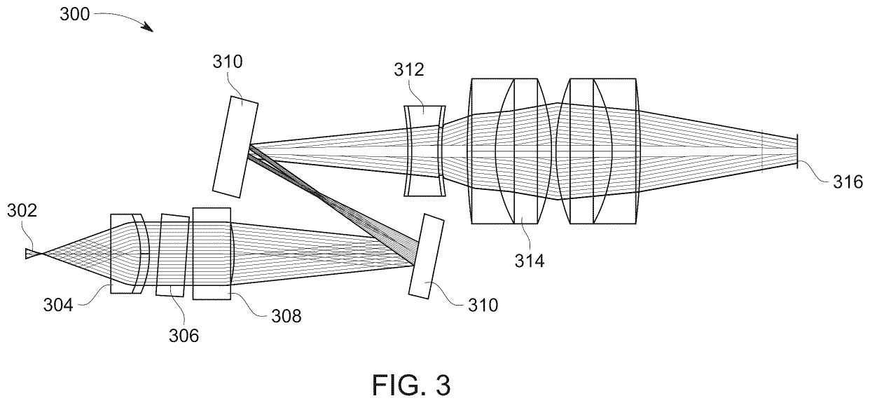

As described above, it will be appreciated that a variety of implementations of lens 208 are available that provide different focusing and light collection characteristics. For example, FIG. 3 provides an example implementation of an optical architecture useful for analyzing a sample contained in a package (e.g., a bag, bottle, etc.), where the optical architecture comprises some components of the optical system 200 (see FIG. 2) and other components that provide the characteristics of lens 208 (see FIG. 2), collectively referred to as an optical arrangement 300. In the described example, the optical arrangement 300 includes an element 302 that may include a focusing lens 203 (see FIG. 2) or an output from an optical fiber. Element 302 directs a beam (e.g., produced from light source 149 or laser assembly 201 or a Raman laser 119—see FIGS. 1, 2, and 5) to a collimating lens 304 that produces a substantially collimated beam. In the described example, the collimating lens 304 can be movably mounted such that it can change position along the axis of the optical path. The range of motion includes a range of about 0.1 mm to about 10 mm to allow for a change in spot size on the sample surface to range from about 10 microns to about 10 mm. It will also be appreciated that in some implementations any of the collimating lens 304, a concave focusing lens 312, and/or focusing optics 314, either alone or in combination, may be movably mounted to effect a change in spot size.

The collimating lens 304 directs the substantially collimated beam into an aspheric diffuse ring producing optic 308 configured to produce a light pattern that is radially diffuse. The intensity of the output from the aspheric diffuse ring producing optic 308 is more intense at the outer edge of the resulting pattern than in the center. While this pattern could be projected directly onto a sample surface 316, in practical application it is advantageous to use one or more steering mirrors 310, one or more filters 306, and focusing elements, such as, for example, a concave focusing lens 312 and focusing optics 314, to direct the radially diffuse light pattern onto the sample surface 316.

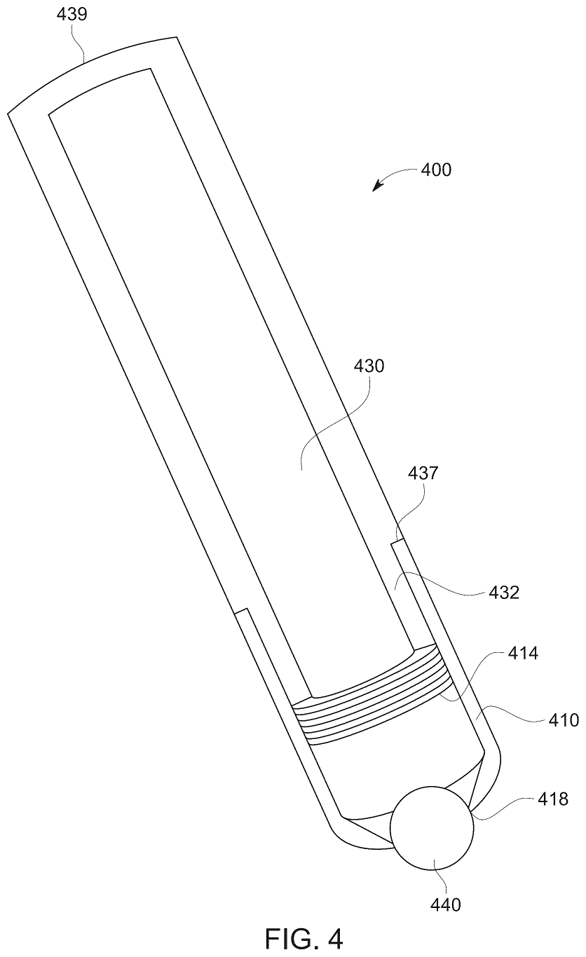

FIG. 4 provides an example of another implementation of the lens 208 (see FIG. 2), wherein this example may be useful for analyzing a fluid or semi-fluid sample. The implementation illustrated in FIG. 4 comprises some components of the optical system 200 and other components that provide characteristics of what is generally referred to as an “immersion probe,” wherein the components are collectively referred herein to as an optical arrangement 400. The implementation illustrated in FIG. 4 comprises a spherical lens 440 seated within a cylindrical probe tip 410 at lens opening 418. A seal between the probe tip 410 and the lens 440 is formed at the opening by any means known in the art, including all forms of welding or braising and the use of epoxies or other adhesives. The probe tip 410 may be any length. Optionally, the probe tip 410 may have threads 414 on its interior surface and may be extended using probe tube 430, which has threaded collar 432 for threading into probe tip 410. A scal is optionally formed between probe tube lip 437 and the distal end of probe tip 410. Further, in the described example, the optical arrangement 400 includes fiber optic coupling 439 that transmits illumination light from the laser assembly 201 (see FIG. 2) as well as scattered light from the sample 260 (see FIG. 2), wherein the sample 260 may include a liquid sample where lens 440 is immersed in the liquid. Also in the described example, the optical arrangement 400 may be configured as a separated element from spectroscopic system 110 (see FIG. 1) where an optical fiber provides optical communication between spectroscopic system 110 and the optical arrangement 400.

It will be appreciated that the examples provided in FIG. 3 and FIG. 4 are for the purposes of illustration and some implementations may include additional or fewer elements as needed for an application. For instance, in some implementations one or more windows, collimating lenses or other optical elements may be employed in applications that utilize a fiber optic coupling or other need for conditioning a beam or protecting internal environments. Therefore, the examples provided in FIG. 3 and FIG. 4 should not be considered as limiting.

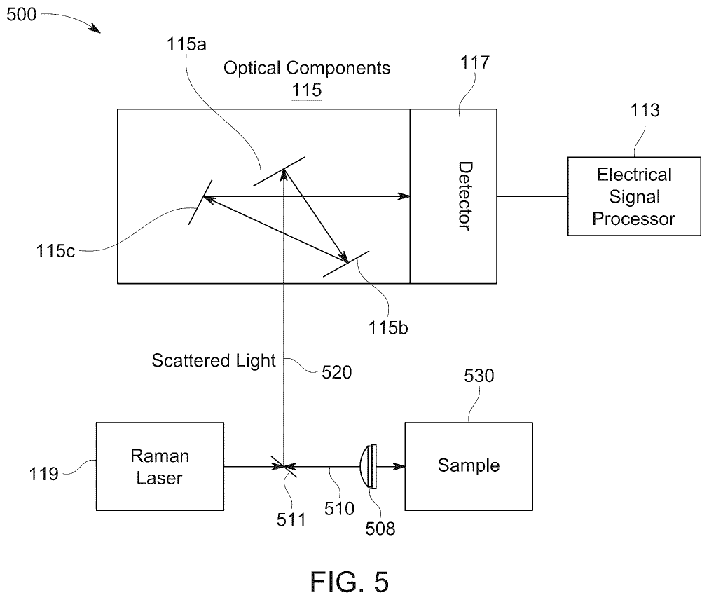

FIG. 5 provides another example of an implementation of an optical architecture comprising optical components of the spectrometer 140 (see FIG. 1), that are otherwise collectively referred to herein as the optical system 500. It will be appreciated that different optical architectures of Raman spectrometer are known in the art and thus the example of FIG. 5, similar to the examples of FIGS. 1 to 4, should not be considered as limiting.

The example of FIG. 5 illustrates one implementation of the light source 149 (see FIG. 1) as a Raman laser 119 comprising a laser source that produces a beam of light that travels along a first optical or beam path 510 (e.g., arrows illustrate direction of travel of the light beam) to a sample 530. Like sample 260 (see FIG. 2), it will be appreciated that sample 530 may include any type of sample of interest to a user which may include substantially dry samples (e.g., a powder, solid material), substantially fluid samples (e.g., a liquid, gas), or some combination thereof (e.g., a gel). In response to the light from the Raman laser 119, the sample 530 produces scattered light along a second optical or beam path 520 (e.g., comprising a Raman portion and a Rayleigh portion of scattered light).

In some implementations, the Raman laser 119 may produce laser power as needed for an application for example, including or between a range of about 250 mW to about 1050 mW, including various subranges therebetween such as the non-limiting subranges described above for the light source 149 and the laser assembly 201. It will also be appreciated that in some implementations, the laser power affects the values of the base value and the bright-max intensity values when the sample 530 is scanned.

FIG. 5 illustrates an architecture that in some implementations directionally controls the first beam path 510 and/or the second beam path 520. In some implementations, the beam paths 510, 520 can be controlled using one or more of turning mirrors, waveguide phase scramblers, various lenses, broadband filters, or selective elements (e.g., mirrors, notch filters, or other elements with substantially reflective characteristics to the wavelength(s) of the beam from the Raman laser 119 and/or substantially transmissive characteristics to a wavelength or wavelength range associated with Raman scattered light from sample 530). In the described example, a selective element 511 is transmissive to the laser wavelengths emitted from the Raman laser 119 allowing the first beam path 510 to be directed to a lens 508 that focuses the beam onto the sample 530. In the described example, the lens 508 may include any type of lens known in the art such as an objective lens or lens architecture such as used in the optical arrangements 300 or 400 (see FIG. 3 and FIG. 4) that focuses the beam onto the sample 530.

Some implementations of the lens 508 include special configurations and characteristics that provides advantages for different types of samples. For example, the lens 508 can collect Raman scattered light and Rayleigh scattered light produced from the sample 530 in response to the beam from the Raman laser 119. The scattered light collected by the lens 508 is directed back from the surface of the sample 530 and travels back along the first beam path 510 to the selective element 511 (e.g., a beam splitter, such as, for example, a dichroic mirror) that directs the scattered light along the second beam path 520. In some implementations, the selective element 511 is substantially reflective to the wavelengths of the Raman scattered light, allowing the second beam path 520 to be directed to additional optical elements that further adjust the path and condition the characteristics of the beam traveling along the second beam path 520. Other optical arrangements are also contemplated for the selective element 511 for directing the scattered light along the second beam path 520.

As illustrated in FIG. 5, the optical system 500 also includes one or more optical components 115 (also referred herein as optical components 115a-115c), which can include one or more of collimating lens and mirrors, filters, such as, for example, a notch filter, diffraction gratings, and/or mirror relays. The scattered light is directed by one or more of optical components 115a-115c onto a detector 117 (an implementation of the detector 147 of FIG. 1). Signal processing and/or digitizing of signals associated with the scattered light that is received by the detector 117 is performed by an electrical signal processor associated with optical system 500, which may be, for example, the electronic signal processor 113, the controller 111, the computing device 120, or a combination thereof. For example, in some implementations, the electrical signal processor 113 may be a suitably programmed microprocessor or application specific integrated circuit including a read-only or read-write memory of any known type which holds instructions and data for spectrometer operation as described herein.

As described above, it will be appreciated that a variety of implementations of the lens 508 are available that provide different focusing and light collection characteristics.

Returning to FIG. 1, the computing device 120 may be a standalone device, a server, internet of things (IoT), a laptop computer, a tablet computer, a netbook computer, a personal computer (PC), a smartphone, a personal digital assistant (PDA), a desktop computer, or any programmable electronic device capable of receiving, sending, and processing data. In some implementations, the computing device 120 includes one or more processors, one or more input/output interfaces, and one or more memory or data storage devices. In some implementations, the computing device 120 also includes one or more input/output devices, such as, for example, a display, a touchscreen, a keyboard, a mouse, or the like, which may be used to provide calibration or setting options to a user for operating the spectroscopic system, to provide analysis results to a user, or a combination thereof.

The computing device 120 may include an I/O interface for communicating with the spectroscopic system 110. The I/O interface may include one or more communication chips, connectors, and/or other hardware and software to govern communications. The I/O interface may include interface circuitry for coupling to one or more components using any suitable interface (e.g., a Universal Serial Bus (USB) interface, a High-Definition Multimedia Interface (HDMI) interface, a Controller Area Network (CAN) interface, a serial Peripheral Interface (SPI) interface, an Ethernet interface, a wireless interface, or any other appropriate interface). For example, the I/O interface may include circuitry for managing wireless communications for the transfer of data to and from the computing device 120. The term “wireless” and its derivatives may be used to describe circuits, devices, systems, methods, techniques, communications channels, etc., that may communicate data through the use of modulated electromagnetic radiation through a nonsolid medium. The term does not imply that the associated devices do not contain any wires, although, in some implementations the associated devices might not. Circuitry included in the I/O interface for managing wireless communications may implement any of a number of wireless standards or protocols, including but not limited to Institute for Electrical Engineers (IEEE) standards including Wi-Fi (IEEE 802.11 family), IEEE 802.16 standards (e.g., IEEE 802.16-2005 Amendment), Long-Term Evolution (LTE) project along with any amendments, updates, and/or revisions (e.g., advanced LTE project, ultra-mobile broadband (UMB) project (also referred to as “3GPP2”), etc.). In some implementations, circuitry included in the I/O interface for managing wireless communications may operate in accordance with a Global System for Mobile Communication (GSM), General Packet Radio Service (GPRS), Universal Mobile Telecommunications System (UMTS), High Speed Packet Access (HSPA), Evolved HPS (E-HPSA), or LTE network. In some implementations, circuitry included in the I/O interface 109 for managing wireless communications may operate in accordance with Enhanced data for GSM Evolution (EDGE), GSM EDGE Radio Access Network (GERAN), Universal Terrestrial Radio Access Network (UTRAN), or Evolved UTRAN (E-UTRAN). In some implementations, circuitry included in the I/O interface for managing wireless communications may operate in accordance with Code Division Multiple Access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. In some implementations, the I/O interface may include one or more antennas (e.g., one or more antenna arrays) for receipt and/or transmission of wire communications. It should be understood that such interfaces and associated communication techniques may be used to provide communication between various components of the analysis system 100, such as, for example, communication with the controller 111 and other components of the analysis system 100.

For example, the controller 111 may additionally include an electronic processor, an input/output interface, and a data storage device (not shown); however, it should be understood that the controller 111 may have additional or fewer components. The controller 111 is suitable for the application and setting, and can include, for example, multiple electronic processors, multiple I/O interfaces, multiple data storage devices, or combinations thereof. In some implementations, some or all of the components included in the controller 111 may be attached to one or more mother boards and enclosed in a housing (e.g., including plastic, metal and/or other materials). In some implementations, some of these components may be fabricated onto a single system-on-a-chip, or SoC (e.g., an SoC may include one or more processing devices and one or more storage devices).

As used herein, “processors” or “electronic processor” refers to any device(s) or portion(s) of a device that process electronic data from registers and/or memory to transform that electronic data that may be stored in registers and/or memory. The electronic processor included in the controller 111 may include one or more digital signal processors (DSPs), application-specific integrated circuits (ASICs), central processing units (CPUs), graphics processing units (GPUs), cryptoprocessors (specialized processors that execute cryptographic algorithms within hardware), server processors, or any other suitable processing devices.

Data storage device(s) included in the controller 111 may include one or more local or remote memory devices such as random-access memory (RAM) devices (e.g., static RAM (SRAM) devices, magnetic RAM (MRAM) devices, dynamic RAM (DRAM) devices, resistive RAM (RRAM) devices, or conductive-bridging RAM (CBRAM) devices), hard drive-based memory devices, solid-state memory devices, networked drives, cloud drives, or any combination of memory devices. In some implementations, the data storage device(s) included in the controller 111 may include memory that shares a die with a processor. In such an embodiment, the memory may be used as a cache memory and may include embedded dynamic random-access memory (eDRAM) or spin transfer torque magnetic random-access memory (STT-MRAM), for example. In some implementations, the data storage device(s) may include non-transitory computer readable media having instructions thereon that, when executed by one or more processors (e.g., the electronic processor included in the controller 111), causes the controller 111 to store various applications and data for performing one or more of the methods described herein or portions described herein.

For example, one or more data storage devices included in the controller 111 may store a modeling program, reactor data, chemometric model data, or a combination thereof. It should be understood that each method described herein may be implemented via one application or multiple applications. Also, the one or multiple applications may be executed via the controller 111, the electronic signal processor 113, the computing device 120, or a combination thereof.

Chemometric model data may include any number of regression analyses or machine learning processes including partial least squares regression (PLS), principal component regression (PCR), least absolute shrinkage and selection operator (LASSO), elastic-net regression, support vector machine (SVM), neural network, or combinations thereof stored on the data storage device.

Reactor data may include one or more physical properties and/or one or more operational parameters. In some implementations, reactor data may include one or more physical properties including a volume of a reactor, a form factor of the reactor, number and types of inlets and outlets, interior surface area, material construction of the one or more reactors, or any combinations thereof. In some implementations, reactor data may include one or more operational parameters including a feed type of a reactor, a method of agitation, pressure, temperature, or any combinations thereof.

In some implementations, volume of the reactor may be between 5 L and 500 L; 10 L and 500 L; 20 L and 500 L; 30 L and 500 L; 40 L and 500 L; 50 L and 500 L; 60 L and 500 L; 70 L and 500 L; 80 L and 500 L; 90 L and 500 L; 100 L and 500 L; 200 L and 500 L; 300 L and 500 L; 400 L and 500 L; or 450 L and 500 L. In some implementations, the volume of the reactor may be no greater than 500 L: no greater than 400 L; no greater than 300 L; no greater than 200 L; no greater than 100 L; no greater than 80 L; no greater than 40 L; or no greater than 20 L. In some implementations, the volume of the reactor may be no less than 5 L; no less than 10 L; no less than 30 L; no less than 50 L; no less than 60 L; no less than 90 L; no less than 150 L; no less than 250 L; no less than 350 L; or no less than 450 L.

In some implementations, feed type to a reactor may include continuous flow, plug flow, batch, or semi-batch.

In some implementations, form factor of the reactor may include a round bottom, a flat bottom, or a conical bottom.

In some implementations, methods of agitation in the one or more reactors may include mixing speed or impeller type. In some implementations, the impeller types may include Rushton, pitch blade and/or marine, helical, and angled pitch-blade.

In some implementations, mixing speeds in the one or more reactors may be between 400 rpm and 800 rpm; 500 rpm and 800 rpm; 600 rpm and 800 rpm; 700 rpm and 800 rpm; 400 rpm and 700 rpm; 400 rpm and 600 rpm; or 400 rpm and 500 rpm. In some implementations, mixing speeds may be no greater than 800 rpm; no greater than 700 rpm; no greater than 600 rpm; or no greater than 500 rpm. In some implementations, mixing speeds may be no less than 400 rpm; no less than 450 rpm; no less than 550 rpm; no less than 650 rpm; or no less than 750 rpm.

In some implementations, the one or more reactors may have an operating pressure between about 50 kPa and about 180 kPa; about 60 kPa and about 180 kPa; about 70 kPa and about 180 kPa; about 80 kPa and about 180 kPa; about 90 kPa and about 180 kPa; about 100 kPa and about 180 kPa; about 110 kPa and about 180 kPa; about 120 kPa and about 180 kPa; about 130 kPa and about 180 kPa; about 140 kPa and about 180 kPa; about 150 kPa and about 180 kPa; about 160 kPa and about 180 kPa; or about 170 kPa and about 180 kPa. In some implementations, the one or more reactors may have an operating pressure between about 90 kPa and about 120 kPa. In some implementations, the one or more reactors may have an operating pressure of no greater than 180 kPa; no greater than 150 kPa; no greater than 120 kPa; no greater than 100 kPa; no greater than 80 kPa. In some implementations, the one or more reactors have a pressure of no less than 50 kPa; no less than 90 kPa; no less than 110 kPa; no less than 140 kPa; or no less than 170 kPa. In some implementations, the one or more reactors are run at atmospheric pressure e.g., about 100 kPa. In some implementations, the reactors are run at higher pressures such as above 180 kPa; above about 500 kPa; above about 1000 kPa; above about 10,000 kPa; above about 35,000 kPa. In some implementations, the reactors have a pressure lower than about 100,000 kPa.

In some implementations, the one or more reactors may have an operating temperature between about 20° C. and about 85° C.; about 30° C. and about 85° C.; about 40° C. and about 85° C.; about 50° C. and about 85° C.; about 60° C. and about 85° C.; about 70° C. and about 85° C.; or about 80° C. and about 85° C. In some implementations, the one or more reactors may have an operating temperature of greater than about 85° C.; greater than about 100° C.; greater than about 120° C.; greater than about 150° C.; greater than about 200° C.; greater than about 300° C.; greater than about 400° C.; or greater than about 500° C. In some implementations the one or more reactors may have an operating temperature less than about 1000° C.; less than about 950° C.; less than about 900° C.; less than about 850° C.; less than about 800° C.; less than about 750° C.; less than about 700° C.; less than about 650° C.; or less than about 600° C.

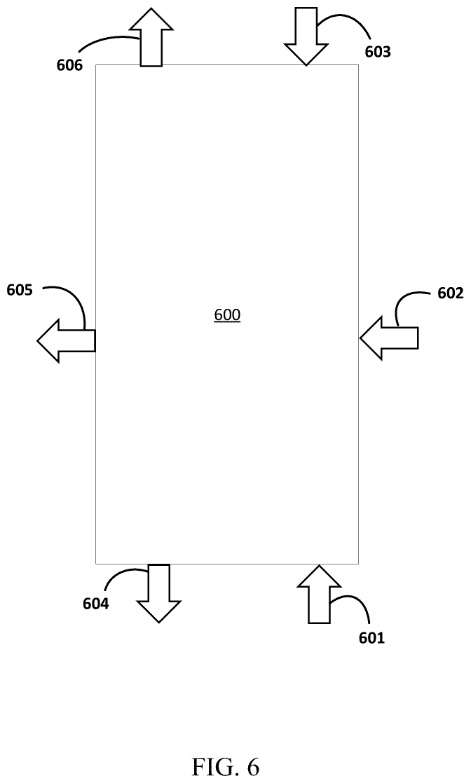

Referring to FIG. 6, an exemplary reactor 600 is schematically depicted for illustrative simplicity to include a plurality of inlets 601, 602, 603 and a plurality of outlets 604, 605, 606. In some implementations, the one or more reactors may include one or more exemplary reactors 600. In some implementations, reactor 600 may include any number of inlets and outlets. In some implementations, reactor 600 may include 1 to 5 inlets and/or 1 to 5 outlets, or combinations thereof. In some implementations, the reactor 600 may include one or more inlets 601 positioned at a bottom portion of the one or more reactors. In some implementations, the reactor 600 may include one or more inlets 602 positioned at a middle portion of the one or more reactors. In some implementations, the reactor 600 may include one or more inlets 603 positioned at a top portion of the one or more reactors. In some implementations, the reactor 600 may include one or more outlets 604 positioned at a bottom portion of the one or more reactors. In some implementations, the reactor 600 may include one or more outlets 605 positioned at a middle portion of the one or more reactors. In some implementations, reactor 600 may include one or more outlets 606 positioned at a top portion of the one or more reactors. In some implementations, the reactor is a bioreactor. In some implementations, the reactor is a chemical reactor.

In some implementations, the material construction of the one or more reactors may include stainless/carbon steel, carbon steel, borosilicate glass, a plastic material such a polypropylene, or any combinations thereof. In some implementations, the material construction includes a disposable bag such as a three-layer plastic foil e.g., for single use bioprocessing.

### II. Exemplary Materials

Exemplary systems and methods involve various materials, such as analytes and matrices. Each of which are discussed below.

### A. Exemplary Target Analyte

Exemplary target analytes may include a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA (e.g., mRNA), DNA, oligonucleotides, recombinant proteins, an edible product, polyols a polymer, or a molecule. In some implementations, the target analyte is a metabolite.

In some implementations, a polymer may have a molecular weight between about 1000 g/mol to about 200,000 g/mol; about 10,000 to about 200,000 g/mol; about 20;000 to about 200,000 g/mol; about 30,000 g/mol to about 200,000 g/mol; about 40,000 g/mol to about 200.00 g/mol; about 50,000 g/mol to about 200,000 g/mol; about 60,000 g/mol to about 200.00 g/mol; about 70,000 g/mol to about 200,000 g/mol; about 80,000 g/mol to about 200,000 g/mol; about 90,000 g/mol to about 200.00 g/mol; about 100,000 g/mol to about 200,000 g/mol; about 120,000 g/mol to about 200,000 g/mol; about 140,000 g/mol to about 200,000 g/mol; about 160,000 g/mol to about 200,000 g/mol; or about 180,000 g/mol to about 200,000 g/mol. In some implementations, a polymer, may have a molecular weight no greater than 200,000 g/mol; no greater than 160,000 g/mol; no greater than 120,000 g/mol; no greater than 80,000 g/mol; no greater than 40,000 g/mol; no greater than 20,000 g/mol; no greater than 10,000 g/mol; no greater than 8,000 g/mol; no greater than 6,000 g/mol; no greater than 4,000 g/mol; or no greater than 2,000 g/mol. In some implementations, a polymer have may have no less than 1000 g/mol; no less than 3,000 g/mol; no less than 5,000 g/mol; no less than 7,000 g/mol; no less than 9,000 g/mol; no less than 15,000 g/mol; no less than 30,000 g/mol; no less than 50,000 g/mol; no less than 70,000 g/mol; no less than 90,000 g/mol; 110,000 g/mol; no less than 150,000 g/mol; no less than 170,000 g/mol; or no less than 190,000 g/mol.

In some implementations, a molecule may have a molecular weight between about 1 g/mol to about 1,000 g/mol; about 10 g/mol to about 1000 g/mol; about 100 g/mol to about 1000 g/mol; about 200 g/mol to about 1000 g/mol; about 300 g/mol to about 1000 g/mol; about 400 g/mol to about 1000 g/mol; about 500 g/mol to about 1000 g/mol; about 600 g/mol to about 1000 g/mol; about 700 g/mol to about 1000 g/mol; about 800 g/mol to about 1000 g/mol; or about 900 g/mol to about 1000 g/mol. In some implementations, a molecule may have a molecular weight no greater than 1000 g/mol; no greater than 800 g/mol; no greater than 600 g/mol; no greater than 400 g/mol; no greater than 200 g/mol; no greater than 80 g/mol; no greater than 60 g/mol; no greater than 40 g/mol; no greater than 20 g/mol; or no greater than 5 g/mol. In some implementations, a molecule may have a molecular weight no less than 1 g/mol; no less than 2 g/mol; no less than 8 g/mol; no less than 10 g/mol; no less than 30 g/mol; no less than 50 g/mol; no less than 70 g/mol; no less than 90 g/mol; no less than 110 g/mol; no less than 130 g/mol; no less than 150 g/mol; no less than 150 g/mol; no less than 170 g/mol; no less than 190 g/mol; no less than 300 g/mol; no less than 500 g/mol; no less than 700 g/mol; or no less than 900 g/mol . . .

In some implementations, exemplary metabolites may include classes including alcohol, amino acids, nucleotides, antioxidants, aldehydes, organic acids or their salts, polyols, vitamins, or any combinations thereof.

In some implementations, exemplary alcohols may include methanol, ethanol, and propanol.

In some implementations, the metabolite is acetic acid or its acetate salt, or acetaldehyde.

In some implementations, the target can be a natural or unnatural amino acid. In some implementations, exemplary amino acids may include one or more of alanine, arginine, asparagine, aspartate, cysteine, glutamate, glutamine, glycine, histidine, methionine, proline, serine, threonine, valine, acids thereof, or salts thereof. In some implementations, the amino acid is glutamine.

In some implementations, exemplary nucleotides may include adenine, thymine, guanine, cytosine, uracil, ribonucleotides, deoxyribonucleotides, or any combinations thereof.

In some implementations, exemplary antioxidants may include vitamin C, vitamin E, selenium, manganese, copper, zinc, carotenoids, (i.e., beta-carotene, lycopene, lutein, and zeaxanthin), catechins, cryptoxanthins, flavonoids, indoles, zoochemicals, polyphenols, anthocyanins, allium sulphur compounds, or any combinations thereof.

In some implementations, exemplary organic acids may include citric acid, acetic acid, lactic acid, formic acid, carboxylic acid, oxalic acid, tartaric acid, malic acid, propionic acid, carbonic acid, butanoic acid, caproic acid, hydron, aspartic acid, or a salt thereof. In some implementations, the organic acid is lactate.

In some implementations, exemplary polyols may include an organic compound with multiple hydroxyl groups (—OH). In some implementations, polyols may include one or more of sugars such as glucose, lactose, sorbitol, maltitol, xylitol, erythritol, isomalt, glycerol, lactitol, mannitol, or hydrogenated starch hydrolysates. In some implementations, polyols may include polymeric polyols such as polyurethanes, polyvinyl alcohol, cellulose, castor oil, or cottonseed oil.

In some implementations, exemplary vitamins may include vitamins A, C, D, E, K, choline, B vitamins (i.e., thiamin, riboflavin, niacin, pantothenic acid, biotin, vitamin B6, B12, and folate and/or folic acid), or any combinations thereof.

In some implementations, exemplary proteins may include albumins, globulins, glutelins, and albuminoids, or any combinations thereof.

In some implementations, exemplary steroids may include androgens, oestrogens, progestrogens, glucocorticoids, or any combinations thereof.

### B. Exemplary Matrices

Exemplary matrices may have an effect on the Raman spectroscopy scan and the quality of the results obtained, these effects are often referred to as matrix effects, as shown below in equation (1):

$$Matrix effect = 100 ( \frac{A ( extract )}{A ( standard )} ) \qquad ( 1 )$$

where, A (extract) is the peak area of the target analyte, when diluted with matrix effect and A (standard) is the peak area of the target analyte in the absence of the matrix.

The matrix effects are factored into the determination of the unknown parameter of the target analyte by building the chemometric model according to the methods and systems described herein.

In some implementations, a matrix effect value close to 100 indicates an absence of matrix influence, whereas a matrix effect less than 100 indicates suppression, and a value larger than 100 indicates matrix enhancement.

In some implementations, exemplary matrices may refer to components of a sample other than the target analyte, as discussed above in detail.

In some implementations, exemplary matrices may include one or more of a serum, a protein, a nutrient, an intermediate of the target analyte, a metabolite, or any combinations thereof. In some implementations, the matrices include a serum.

In some implementations, exemplary matrices does not include the target analyte and may include one or more of a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA (e.g., mRNA), DNA, oligonucleotides, recombinant proteins, an edible product, polyols a polymer, or a molecule. In some implementations, the target analyte is a metabolite.

In some implementations, exemplary matrices may include the target analyte and one or more of a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA (e.g., mRNA), DNA, oligonucleotides, recombinant proteins, an edible product, polyols a polymer, or a molecule. In some implementations, the target analyte is a metabolite.

### III. Exemplary Methods of Operation

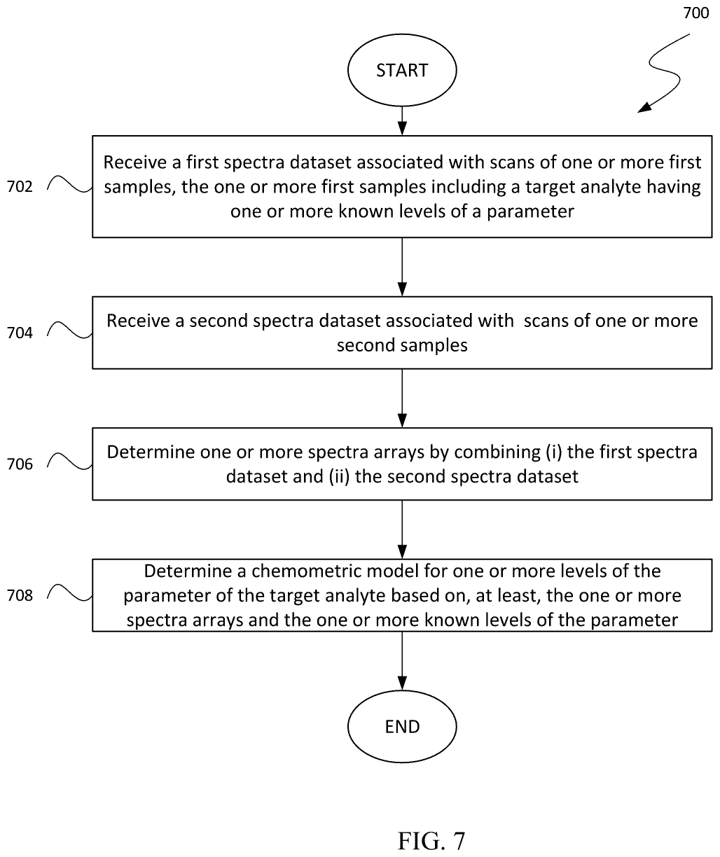

Referring now to FIG. 7, a flowchart illustrates a process 700 for determining measurement parameters of a sample compound, in accordance with some implementations of the present disclosure. Process 700 may be implemented using the spectrometer 140, as described above. The process 700 is described herein as being performed via the controller 111. However, it should be understood that the process 700 may be performed by one or more software and/or hardware components in various combinations and configurations. As illustrated in FIG. 7, the process 700 may include operations 702, 704, 706, or 708. In some implementations, the process 700 is performed in the order as illustrated in FIG. 7. In some implementations, the process 700 may be performed in one or more orders other than what is illustrated in FIG. 7.

In some implementations, process 700 may begin by performing scans of one or more samples, as discussed in detail above. The sample is scanned using, at least, the spectrometer 140, as described above in FIGS. 1-5. The spectrometer 140 directs a Raman laser beam (e.g., produced from the light source 149 or the laser assembly 201 or the Raman laser 119—see FIGS. 1, 2, and 5), as described above, onto a surface or focal point of a sample. For example, the Raman laser beam can be directed into a solution containing any of the target analytes described above. The resulting scattered light is directed back through the selective element 511 and the scattered light travels along the scattered light path 520 and through the optical components 115 onto the detector 117. The resulting Raman spectrum of the sample is received by the detector 117, and signal processing and/or digitizing of the received spectrum is handled by the electrical signal processors 113.

In some implementations, a scan of the one or more samples is captured from 1 millisecond (ms) to 20 seconds exposure time. In some implementations, the scans of the one or more samples captures both the bright and dark Raman spectra of the sample.

In some implementations, the parameter may include concentration, fluorescence, refractive index, mass spectra, electrochemical behavior, or combinations thereof. As used herein the “level” of the parameter is a qualitative or quantitative value or quantity corresponding to the parameter. For example, the parameter may be a concentration where the level is the value of the concentrations such as in grams per milliliter. As another example, the parameter may be a concentration where the level is selected from a qualitative value such as low, medium, and high.

In operation 702, the controller 111 receives a first spectra dataset associated with scans of one or more first samples, the one or more first samples including at least one target analyte described herein, the target analyte having one or more known levels of a parameter.

In some implementations, the electrical signal processor 113 determines the Raman spectra data from scans of the one or more first samples. The electrical signal processor 113 determines the Raman spectra data from the received Raman spectra data and communicates the Raman spectra data to controller 111. In some implementations, controller 111 generates a spectrum representation of the received Raman spectra data of the sample. The spectrum representation can be a visual plot (e.g., a graph) or table shown by the display 112, or the spectrum representation can be an array of values stored in the data storage device(s) which can be used by components of the controller 111, such as the modeling program.

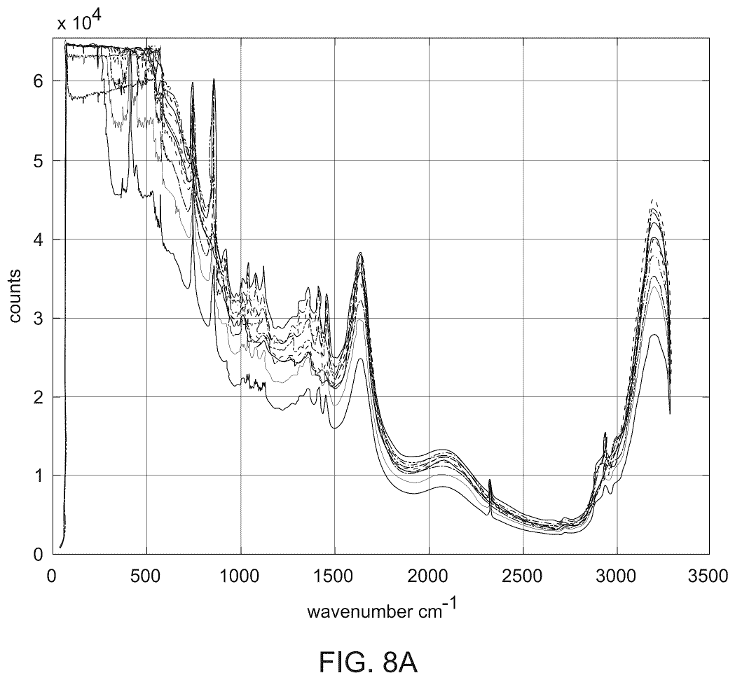

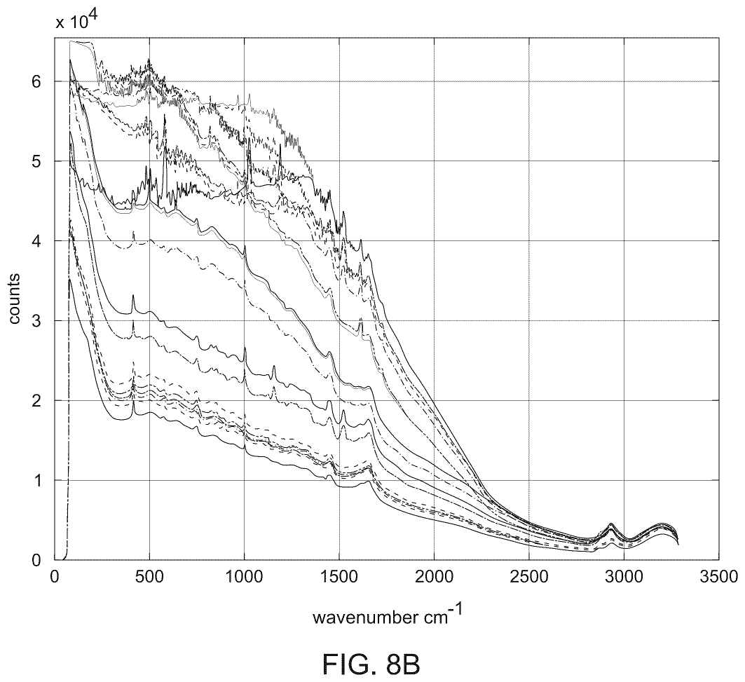

As will be discussed below, an example of the Raman shift wavenumbers is shown in FIG. 8A.

In some implementations, the controller 111 analyzes the Raman shift wavenumbers, as illustratively shown in FIG. 8A, and the controller 111 identifies one or more Raman shift wavenumbers associated with the first spectra dataset. As depicted in FIG. 8A, the first spectra dataset may include several Raman scans each having wavenumbers in a range, here between about 0 (the laser wavelength) and up to about 3300 cm⁻¹. In some implementations, the controller 111 stores the one or more Raman shift wavenumbers associated with the first spectra dataset on one or more data storage device(s) (e.g., as included in the controller 111). In some implementations, the controller 111 communicates a set of program instructions to the user interface 114 instructing user interface 114 to display the one or more Raman shift wavenumbers associated with the first spectra dataset on the display 112.

FIG. 8A illustrates the Raman shift wavenumbers of the first spectra dataset associated with one or more reactors collected from scans of one or more first samples, where the one or more first samples include a target analyte having one or more known levels of a parameter.

In some implementations, pre-processing operations are applied to the first spectra dataset and the second spectra dataset, as described below in greater detail. In some implementations, the pre-processing operations are applied to the first spectra dataset and the second spectra dataset before one or more spectra arrays are determined by combining the (i) the first spectra dataset and (ii) the second spectra dataset.

Returning to FIG. 7, in some implementations, the controller 111 applies one or more pre-processing operations to the first spectra dataset. In some implementations, the one or more pre-processing operations include region selection, spectra, averaging, convolution filtering, 1^{st} derivative, 2^{nd} derivative, standard normal variate, multiplicative scatter correction, background removal, or combinations thereof. In some implementations, the controller 111 stores the pre-processed first spectra dataset on one or more data storage device(s) (e.g., in the controller 111).

In operation 704, the controller 111 receives a second spectra dataset associated with scans of one or more second samples.

In some implementations, the electrical signal processor 113 determines the Raman spectra data from scans of the one or more second samples. The electrical signal processor 113 determines the Raman spectra data from the received Raman spectra data and communicates the Raman spectra data to controller 111. In some implementations, controller 111 generates a spectrum representation of the received Raman spectra data of the sample. The spectrum representation can be a visual plot (e.g., a graph) or table shown by the display 112, or the spectrum representation can be an array of values stored in the data storage device(s) which can be used by components of the controller 111, such as the modeling program.

In some implementations, the controller 111 determines one or more Raman shift wavenumbers associated with the second spectra dataset. In some implementations, the controller 111 stores the one or more Raman shift wavenumbers associated with the second spectra dataset on one or more data storage device(s) (e.g., in the controller 111).

As will be discussed below, an example of the Raman shift wavenumbers for the second spectra dataset is shown in FIG. 8B.

In some implementations, the controller 111 communicates a set of program instructions to user interface 114 instructing user interface 114 to display the one or more Raman shift wavenumbers associated with the second spectra dataset on display 112.

FIG. 8B illustrates the Raman wavenumbers of the second spectra dataset associated with one or more reactors collected from scans of one or more second samples, where the one or more second samples include a matrix.

Returning to FIG. 7, in some implementations, the controller 111 applies one or more pre-processing operations to the second spectra dataset. In some implementations, the one or more pre-processing operations include region selection, spectra, averaging, convolution filtering, 1st derivative, 2nd derivative, standard normal variate, multiplicative scatter correction, background removal, and combinations thereof. In some implementations, the controller 111 stores the pre-processed second spectra dataset on one or more data storage device(s) (e.g., in the controller 111).

In operation 706, the controller 111 determines one or more spectra arrays by combining (i) the first spectra dataset and (ii) the second spectra data.

In some implementations, the controller 111 retrieves the first spectra dataset that includes pre-processing and the second spectra dataset that includes pre-processing as stored in one or more data storage device(s). In some implementations, the pre-processing operations applied to the first spectra dataset are the same pre-processing operations applied to the second spectra dataset. In some implementations, the pre-processing operations applied to the first spectra dataset are not the same pre-processing operations applied to the second spectra dataset.

For example, the first spectra dataset may be illustrated as shown below in Table 1:

TABLE 1

| Target | Wavenumbers | | | |
|---|---|---|---|---|
| Analyte ↓ | (cm⁻¹) → | 1000 | 1001 | 1002 |
| 1 | Counts | 30000 | 30600 | 30000 |
| 2 | | 24000 | 30000 | 27000 |
| 3 | | 33000 | 36000 | 42000 |

For example, the second spectra data may be illustrated as shown below in Table 2:

TABLE 2

| Matrices | Wavenumbers | | | |
|---|---|---|---|---|
| ↓ | (cm⁻¹) → | 1000 | 1001 | 1002 |
| 1 | Counts | 20000 | 21000 | 22000 |
| 2 | | 25000 | 24000 | 23000 |
| 3 | | 22000 | 21000 | 20000 |

In some implementations, the controller 111 determines the proportional values of the first spectra dataset and the second spectra dataset. In some implementations, the proportional values of the first spectra dataset and the second spectra dataset are determined by applying a value between 0 and 1 to the one or more counts of the first spectra dataset and the second dataset. For example, if one count associated with the first spectra dataset is 30,000 and a value of 0.9 is applied to the count of 30,000 associated with the first spectra dataset, then the determined proportional value is 27,000. In some implementations, the proportional value for the first spectra dataset may include 0, 0.1, 0.2, 0.3, 0.4, 0.5, 0.6, 0.7, 0.8, 0.9, and 1. In some implementations, the controller 111 determines a proportional value of the first spectra data between 0 and 1. In some implementations, the proportional value for the second spectra dataset may include 0, 0.1, 0.2, 0.3, 0.4, 0.5, 0.6, 0.7, 0.8, 0.9, and 1. In some implementations, the controller 111 determines a proportional value of the second spectra data between 0 and 1. For example, if one count associated with the second spectra dataset is 20,000 and a value of 0.9 is applied to the count of 20,000 associated with the second spectra, then the determined proportional value is 18,000. In some implementations, the proportional values of the first spectra dataset and the second spectra dataset equal 1. For example, if the proportional value of the first spectra dataset is 0.7, then the proportional value of the second spectra dataset is 0.3. In another example, if the proportional value of the first spectra dataset is 0.2, then the proportional value of the second spectra dataset is 0.8. As discussed above, the combined proportional values of the first spectra dataset and the second spectra dataset equal 1.

In some implementations, the controller 111 sums the values of the first spectra dataset and the values of the second spectra dataset. In some implementations, the controller 111 sums the values of the first spectra dataset based on, at least, a proportional value between 0 and 1 and the second spectra dataset based on, at least, a proportional value between 0 and 1. For example, the controller 111 sums the values of the first spectra dataset, as shown in Table 1, and the values of the second spectra dataset, as shown in Table 2, having proportional values as shown below in Table 3, wherein the values in Table 3 are an example of a spectra array:

TABLE 3

| Target | Wavenumbers | | | |
|---|---|---|---|---|
| Analyte | (cm⁻¹) --> | 1000 | 1001 | 1002 |
| 1 | Counts: 0.1 | 21000 | 21960 | 22800 |
| 2 | Target + 0.9 | 24900 | 24600 | 23400 |
| 3 | Matrix | 23100 | 22500 | 22200 |

In some implementations, the controller 111 implements a set of program instructions to apply a weighted average to the summation of the first spectra dataset and the second spectra dataset. That is, the controller 111 implements a set of program instructions to apply a weighted average to the summed value of the proportional value of the first spectra dataset and the proportional value of the second spectra dataset. In some implementations, the weighted average includes a weight from 0 to 6. In some implementations, a weighted average is applied to the first spectra dataset and the second spectra data by multiplying each data point associated with the first spectra dataset and the second spectra dataset by a weight between 0 and 6 and summing the products of each data point associated with the first spectra dataset and the second spectra dataset, respectively. Next the total weights for each data point associated with the first spectra dataset and the second spectra dataset, respectively, as summed. Then, the summed product of each data point associated with the first spectra dataset and the second spectra dataset is divided by the summed total weight and the result is a weighted average of the summed value of the proportional values of the first spectra dataset and the second spectra dataset, respectively.

In some implementations, the controller 111 generates one or more spectra arrays based on, at least, the weighted average of the summation of the first spectra dataset and the second spectra dataset.

In operation 708, the controller 111 determines a chemometric model for one or more levels of the parameter of the target analyte(s) based on, at least, the one or more spectra arrays and the one or more known levels of the parameter.

In some implementations, the controller 111 builds the chemometric model based on the one or more spectra arrays and the one or more known levels of the parameter of the target analyte(s). In some implementations, the controller 111 builds the chemometric model based on, at least, a partial least squares regression (PLS) model, a principal component regression (PCR) model, a least absolute shrinkage model and selection operator (LASSO) model, an elastic net regression model, a support vector machines (SVM) model, or a neural network model.

In some implementations, the chemometric model includes one or more physical properties, as described above in detail, of the one or more reactors that are associated with the first spectra dataset and the second spectra dataset. In some implementations, the controller 111 further builds the chemometric model based on one or more operational parameters, as described above in detail, of the one or more reactors associated with the first spectra dataset and the second spectra dataset.

In some implementations, the controller 111 receives data from the chemometric model and identifies a prediction correlation (R2P) value and the one or more levels of the parameter of the target analyte(s). In some implementations, the one or more levels of the parameter includes the error rate (e.g., concentration) provided by the chemometric model. In some implementations, the acceptable criteria for error rate are represented by the root mean square error of prediction (RMSEP) value of the chemometric model, provided that the RMSEP value is below 1 g/L.

In some implementations, the controller 111 determines the one or more levels of the parameter of the target analyte(s) and communicates a set of program instructions to the user interface 114 instructing the user interface 114 to display the one or more levels of the parameter of the target analyte(s) to a user of the analyzer 100. In some implementations, the controller 111 stores the one or more levels of the parameter of the target analyte(s) on one or more data storage device(s) (e.g., in the controller 111).

In some implementations, the controller 111 determines a level of concentration of the target analyte(s) in a third example based on, at least, the determined chemometric model and a spectra dataset from a scan of the third sample.

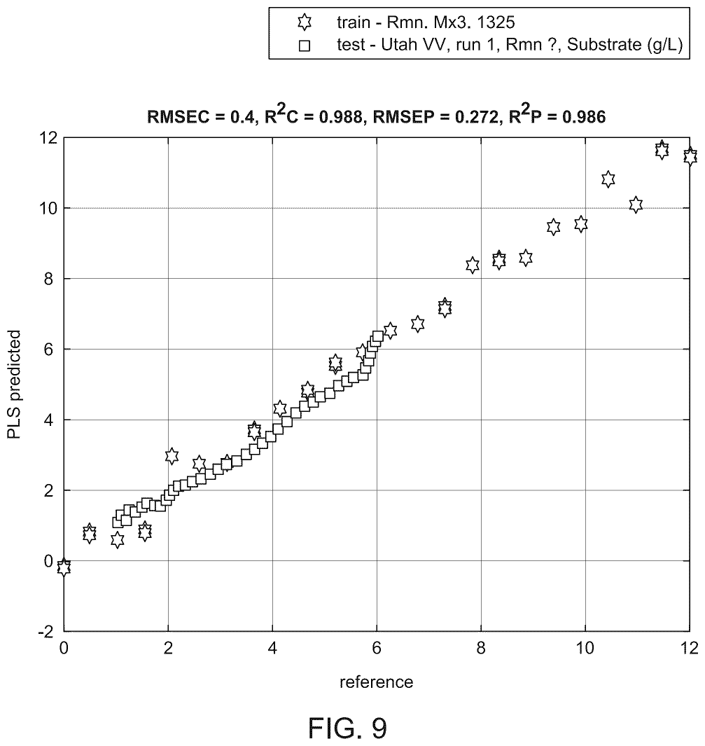

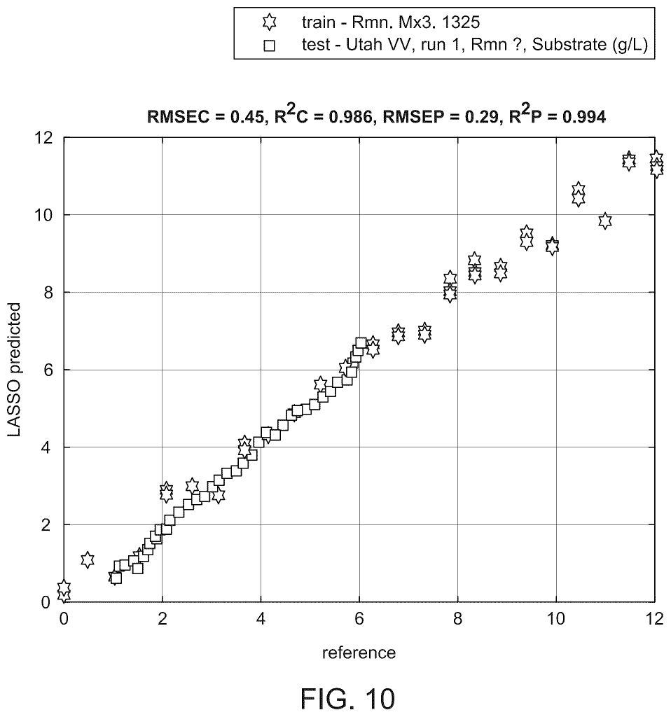

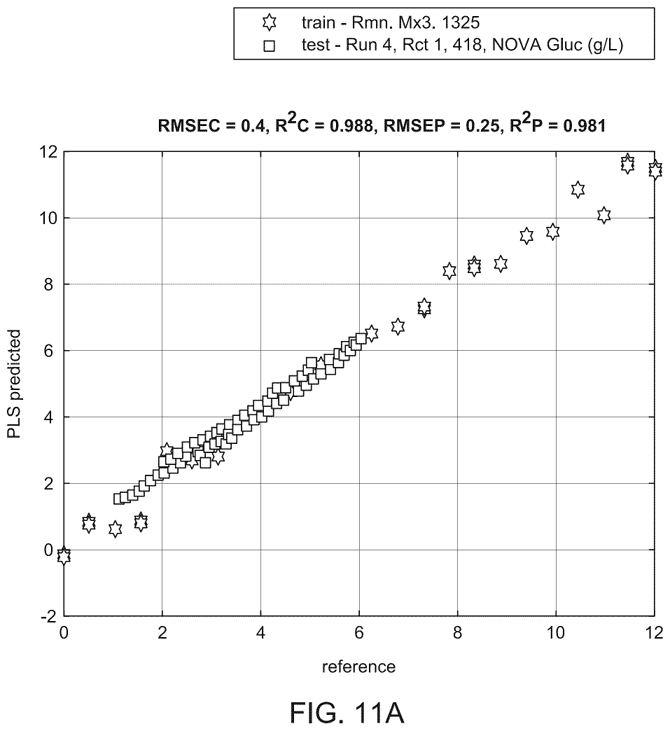

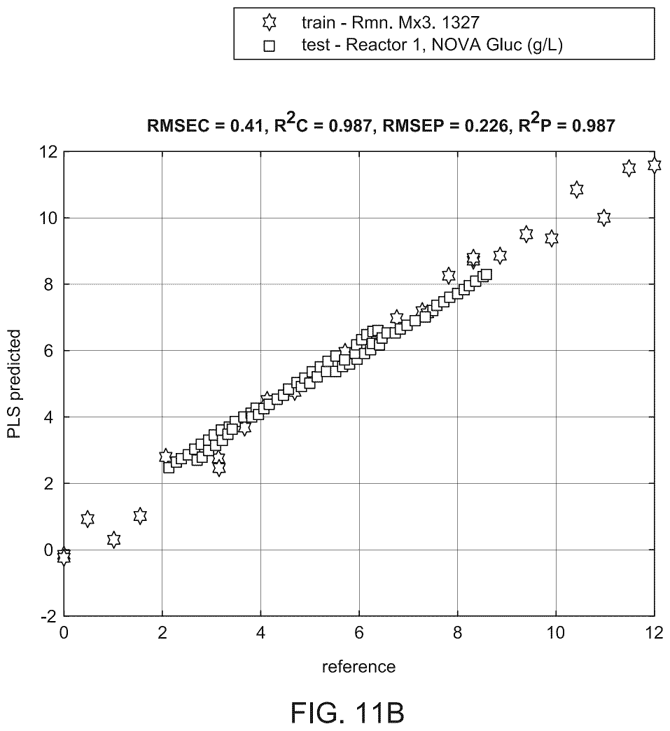

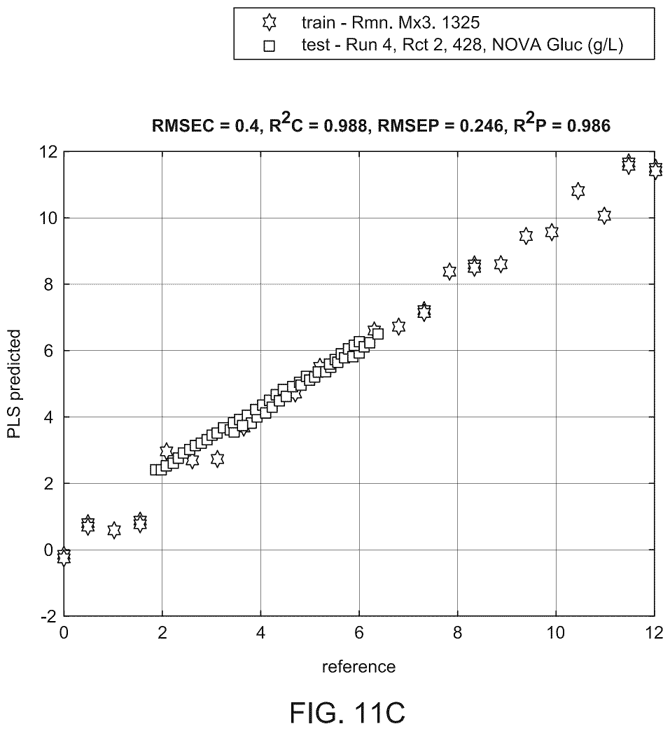

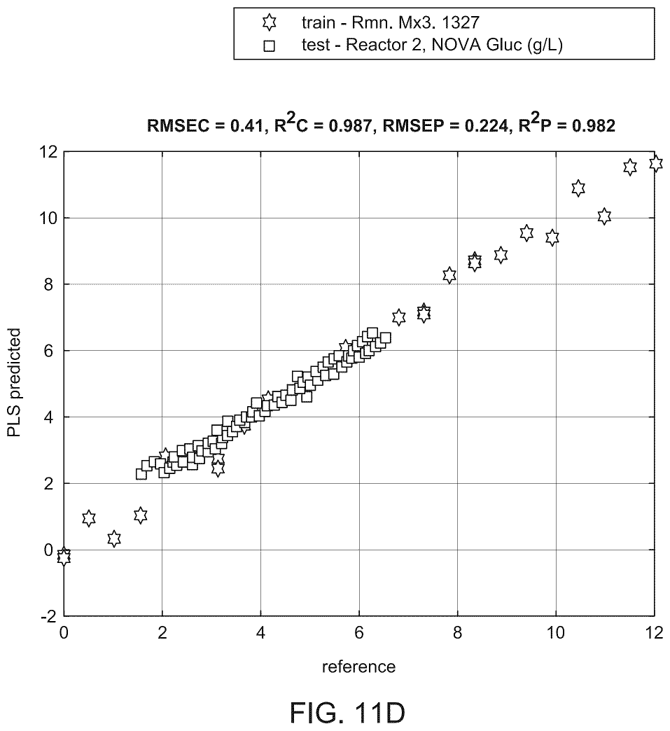

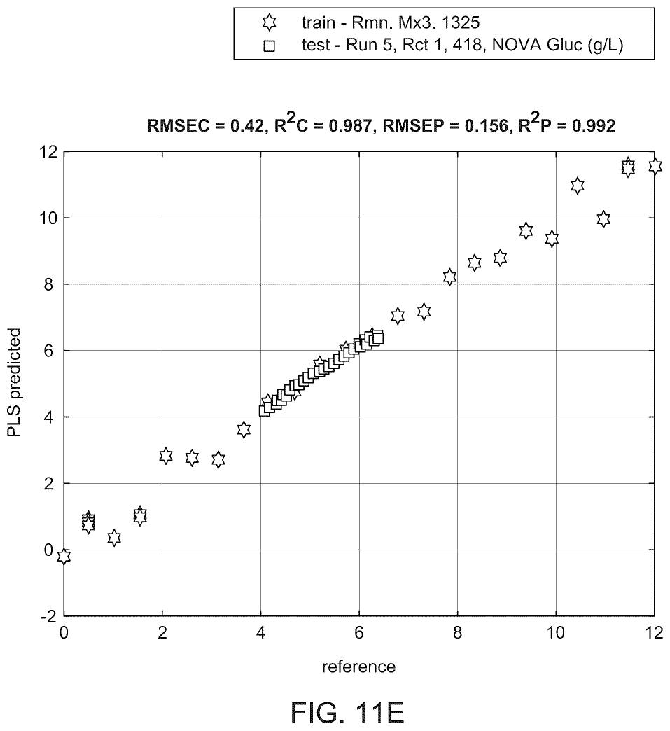

In some implementations, the controller 111 determines the chemometric model for a level of a parameter of the target analyte(s) based on, at least, the one or more spectra arrays and the one or more known levels of the parameter. As will be discussed below, an example of the chemometric model is shown below in FIG. 9 (illustrating a first chemometric model) and FIG. 10 (illustrating a second chemometric model). These plots and similar ones presented in FIGS. 11A-11E show the known glucose concentrations (mg/mL) on the abscissa and the model predicted glucose concentrations (mg/mL or g/L) on the ordinate. Two sets of data are plotted: the training data and the test data.

FIG. 9 illustrates performance of the first chemometric model based on PLS. FIG. 9 further illustrates a R2P value of 0.986 and a RMSEP value (i.e., error rate) of 0.272 g/L. Based on this illustration, it was observed that the first chemometric model of FIG. 9 has an acceptable error rate below 1 g/L for the target analyte used.

FIG. 10 illustrates performance of the second chemometric model based on LASSO. FIG. 10 further illustrates a R2P value of 0.994 and a RMSEP value (i.e., error rate) of 0.29 g/L. Based on this illustration, it was observed that the second chemometric model of FIG. 10 has an acceptable error rate below 1 g/L for the target analyte used.

Further regarding FIGS. 9 and 10, the test data were collected under the same conditions, i.e., the same reactor operated under the same conditions.

### IV. Experimental Data

### A. Exemplary Chemometric Models

In some implementations, the experimental data described below in this section includes, at least, any one of the methods of operation described above in FIGS. 7-8B. The performance of an exemplary PLS model is illustrated by FIGS. 11A-11E, where the amount of glucose was monitored in various biochemical reactions. The exemplary PLS model is the same model used with reference to FIG. 9. The model is based on PLS and trained using one or more exemplary spectra arrays and one or more exemplary known levels of a parameter of the target analyte used.

FIG. 11A illustrates the performance of the exemplary chemometric model for predicting glucose levels in a biochemical reaction including an antibody titer and a first reactor having physical properties including a fed batch, 5 L, and glass material. It was observed that FIG. 11A illustrates a R2P value of 0.981 and a RMSEP value (i.e., error rate) of 0.25 g/L. It was further observed that the RMESEP value was within the acceptable range below 1 g/L.

FIG. 11B illustrates the performance of the exemplary chemometric model for predicting glucose levels in a second reactor, where the rest of the conditions are the same as described with reference to FIG. 11A. It was observed that FIG. 11B illustrates a R2P value of 0.987 and a RMSEP value (i.e., error rate) of 0.226 g/L. It was further observed that the RMSEP value was within the acceptable range below 1 g/L.

FIG. 11C illustrates the performance of the exemplary chemometric model for predicting glucose levels in a reactor (different from the reactors described with reference to FIGS. 11A and 11B). The conditions are the same as described with reference to FIGS. 9A and 9B. FIG. 11C illustrates the performance of a chemometric model built from a first spectra dataset collected from one or more samples, the one or more samples include antibodies as the target analyte and one or more reactors having physical properties including a fed batch, 5L, and glass material. It was observed that FIG. 11C illustrates a R2P value of 0.986 and a RMSEP value (i.e., error rate) of 0.246 g/L. It was further observed that the RMSEP value was within the acceptable range below 1 g/L.

FIG. 11D illustrates the performance of the exemplary chemometric model for predicting glucose levels in a reactor (different from the reactors described with reference to FIGS. 11A, 11B, and 11C) FIG. 11D illustrates a R2P value of 0.982 and a RMSEP value (i.e., error rate) of 0.224 g/L. It was further observed that the RMSEP value was within the acceptable range below 1 g/L.

FIG. 11E illustrates the performance of the exemplary chemometric model for predicting glucose levels in a reactor. The reactor used had physical properties including a hybrid perfusion, 500 L, and dyna drive. It was observed that FIG. 11C illustrates a R2P value of 0.992 and a RMSEP value (i.e., error rate) of 0.156 g/L. It was further observed that the RMSEP value was within the acceptable range below 1 g/L.

Returning to FIG. 9, the performance of the first chemometric model predicting glucose levels in a reactor is illustrated. The reactor used was similar to the reactor used with reference to FIG. 11E: including a hybrid perfusion, 500 L, and dyna drive. The performance is described above.

### B. Chemometric Modelling Data

For training the models described above, samples containing the target analyte and samples containing matrix materials were collected as described herein, where some additional details are described below.

For the target analyte, samples containing glucose, glutamine, and lactate were prepared in water. These were then scanned to obtain their Raman spectra, which spectra are depicted in FIG. 8A. In order to cover the concentration levels of interest, a Uniform Design was used. The methods described herein do not require a designed experiment approach and can use randomly selected concentrations. The ranges for the designed experiments are indicated in Table 4 and the actual concentration values are listed in Table 5 below:

TABLE 4

| | Glucose | | Glutamine | | Lactate | |
|---|---|---|---|---|---|---|
| | (mg/mL) | mM | (mg/mL) | mM | (mg/mL) | mM |
| Lower | 0 | 0 | 0 | 0 | 0 | 0 |
| Control | | | | | | |
| Limit | | | | | | |
| Upper | 12 | 66.6 | 2.5 | 17.1 | 20 | 224.6 |
| Control | | | | | | |
| Limit | | | | | | |

TABLE 5

| | Glucose | Glutamine | Lactate |
|---|---|---|---|
| Run Order | (mg/mL) | (mg/mL) | (mb/mL) |
| 4 | 1.0 | 2.3 | 2.6 |
| 5 | 6.8 | 0.0 | 9.6 |
| 13 | 8.3 | 1.6 | 7.0 |
| 10 | 9.4 | 1.1 | 7.8 |
| 14 | 0.5 | 0.5 | 15.7 |
| 8 | 11.5 | 2.1 | 5.2 |
| 6 | 4.2 | 0.1 | 17.4 |
| 22 | 5.2 | 1.7 | 14.8 |
| 12 | 10.4 | 0.2 | 1.7 |
| 17 | 8.9 | 0.4 | 13.9 |
| 16 | 11.0 | 1.4 | 18.3 |
| 3 | 7.8 | 1.8 | 0.0 |
| 20 | 5.7 | 0.7 | 4.3 |
| 19 | 2.1 | 2.0 | 19.1 |
| 15 | 9.9 | 2.4 | 16.5 |
| 24 | 3.7 | 2.5 | 8.7 |
| 23 | 4.7 | 1.3 | 3.5 |
| 9 | 0.0 | 1.5 | 10.4 |
| 18 | 6.3 | 2.2 | 12.2 |
| 7 | 7.3 | 1.0 | 20.0 |
| 1 | 12.0 | 0.8 | 11.3 |
| 21 | 3.1 | 0.9 | 0.9 |
| 11 | 1.6 | 0.3 | 6.1 |
| 2 | 2.6 | 1.2 | 13.0 |

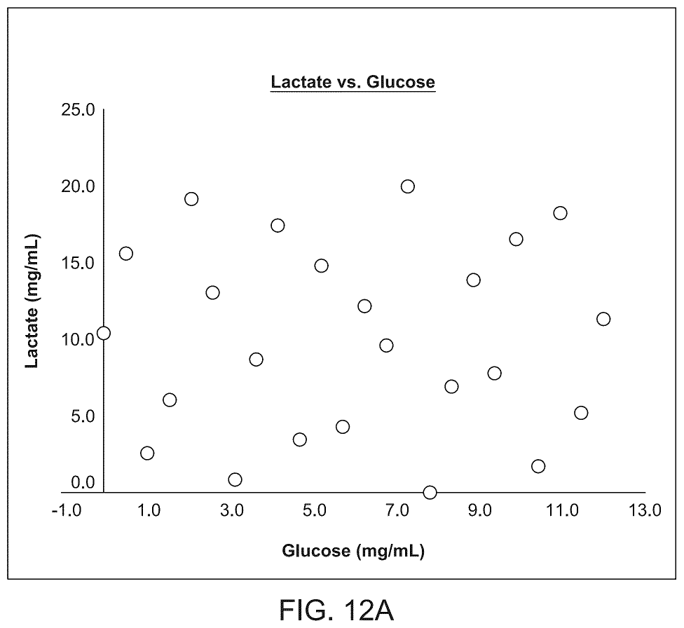

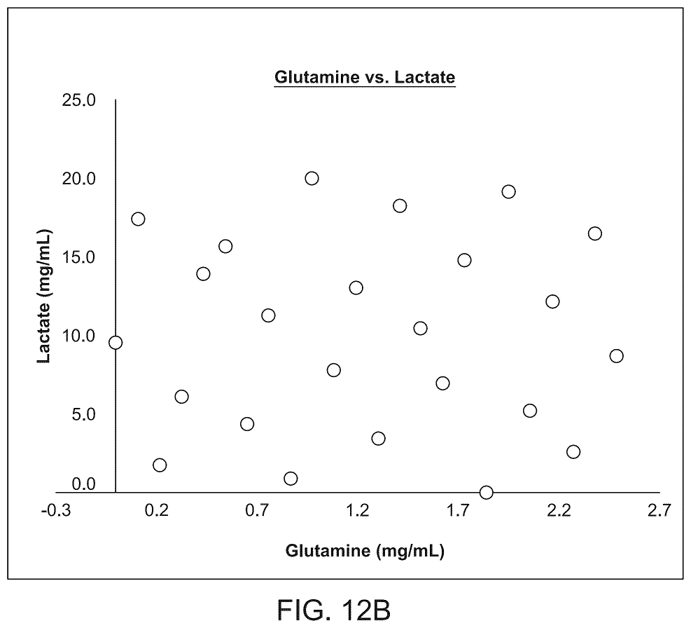

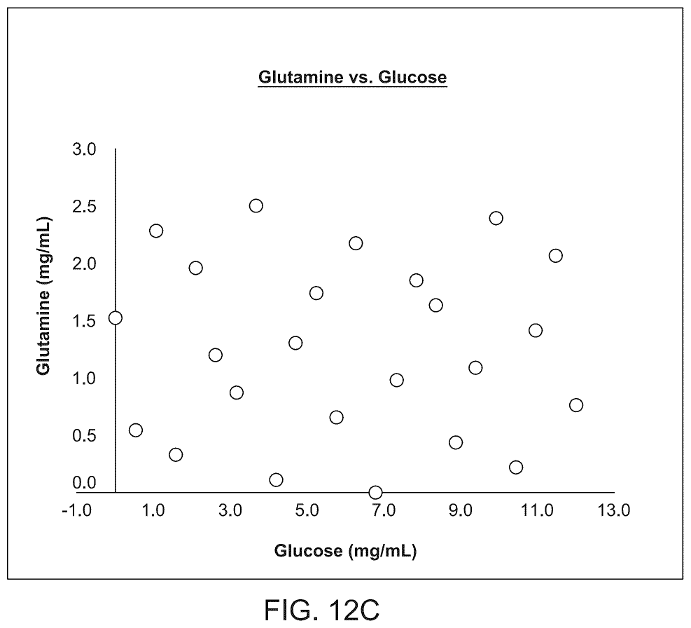

FIGS. 12A, 12B, and 12C are plots illustrating the chemical space covered in this design. FIG. 12A shows lactose to glucose concentration levels. FIG. 12B shows glutamine to Lactate concentrations. FIG. 12C shows glutamine to glucose concentrations. The models were trained using the target analyte, glucose. The models predict the glucose concentration. Training may also be performed for the concentrations of lactate and/or glutamine, in which the models would predict the lactate and/or glutamine concentrations.

Raman spectra of various serum samples were measured and are shown in FIG. 8B. All of the serums were from Thermo Fisher Scientific (GIBCO®) and were scanned in their concentrated form. The serum sample identities are listed in Table 6:

TABLE 6

| | | | Scan | | Laser | |
|---|---|---|---|---|---|---|
| Spectra | Serum | | time | | Power | Probe |
| # | Type | Origin | (ms) | averages | (mW) | Measurement |
| 1 | horse | Country | 1250 | 30 | 450 | Immersion |
| | serum | 1 | | | | |
| 2 | horse | Country | 1250 | 30 | 450 | Immersion |
| | serum | 1 | | | | |
| 3 | horse | Country | 1250 | 30 | 450 | Immersion |
| | serum | 1 | | | | |
| 4 | fetal | Country | 1250 | 30 | 450 | Immersion |
| | bovine | 2 | | | | |
| | serum | | | | | |
| 5 | fetal | Country | 1250 | 30 | 450 | Immersion |
| | bovine | 2 | | | | |
| | serum | | | | | |
| 6 | fetal | Country | 1250 | 30 | 450 | Immersion |
| | bovine | 2 | | | | |
| | serum | | | | | |
| 7 | bovine | Country | 1250 | 30 | 450 | Immersion |
| | serum | 1 | | | | |
| 8 | bovine | Country | 1250 | 30 | 450 | Immersion |
| | serum | 1 | | | | |
| 9 | bovine | Country | 1250 | 30 | 450 | Immersion |
| | serum | 1 | | | | |
| 10 | fetal | Country | 1250 | 30 | 450 | Immersion |
| | bovine | 2 | | | | |
| | serum | | | | | |
| 11 | fetal | Country | 1250 | 30 | 450 | Immersion |
| | bovine | 2 | | | | |
| | serum | | | | | |
| 12 | fetal | Country | 1250 | 30 | 450 | Immersion |
| | bovine | 2 | | | | |
| | serum | | | | | |
| 13 | fetal | Country | 1250 | 30 | 450 | Immersion |
| | bovine | 3 | | | | |
| | serum | | | | | |
| 14 | fetal | Country | 1250 | 30 | 450 | Immersion |
| | bovine | 3 | | | | |
| | serum | | | | | |
| 15 | fetal | Country | 1250 | 30 | 450 | Immersion |
| | bovine | 3 | | | | |
| | serum | | | | | |
| 16 | fetal | Country | 1000 | 30 | 450 | Immersion |
| | bovine | 3 | | | | |
| | serum | | | | | |
| 17 | horse | Country | 1250 | 30 | 450 | Through vial |
| | serum | 1 | | | | |
| 18 | horse | Country | 1250 | 30 | 450 | Through vial |
| | serum | 1 | | | | |
| 19 | horse | Country | 1250 | 30 | 450 | Through vial |
| | serum | 1 | | | | |
| 20 | horse | Country | 1250 | 30 | 450 | Through vial |
| | serum | 1 | | | | |
| 21 | horse | Country | 1250 | 30 | 450 | Through vial |
| | serum | 1 | | | | |
| 22 | horse | Country | 1250 | 30 | 450 | Through vial |
| | serum | 1 | | | | |
| 23 | horse | Country | 1250 | 30 | 450 | Through vial |
| | serum | 1 | | | | |
| 24 | horse | Country | 1250 | 30 | 450 | Through vial |
| | serum | 1 | | | | |
| 25 | horse | Country | 1250 | 30 | 450 | Through vial |
| | serum | 1 | | | | |

Each of the 24 Raman spectra from the chemical mixtures listed in Table 5 can be combined by summing each of the counts at each wavelength, in increments of 0.1 pixels/cm, which results in an average spectral resolution of 6.5 cm⁻¹ in portions from 0 to 1. Increments of 0.1 may be combined with each of the 25 Raman spectra from the serum samples of Table 6. This provides 6,600 combined Raman spectra (24 chemical mixtures×25 serum samples×11 different weights from 0 to 1) with corresponding known levels of glucose. To lower the computing time needed, a smaller dataset was used where 1800 combined Raman spectra (24 chemical mixtures×25 serum samples×3 different weights from 0 to 1) were used. The improvement in the models by increasing the dataset size above 1800 is expected to be minimal such that, for example, using 5000 combined Raman spectra (24 chemical mixtures×25 serum samples×5 different weights from 0 to 1) would also be expected to provide a very similar model. The glucose concentrations were appropriately scaled based on the weight applied to the chemical mixture spectra when combined with the serum spectra. The combined Raman spectral data and glucose concentrations were then used to train the PLS model or the LASSO model as previously described.

### C. Preprocessing and Chemometric Model Construction

The combined Raman spectra data as described above was used in chemometric modelling. In addition, the combined Raman spectra data were pre-processed. 1st derivative and standard normal variate (SNV) were applied to the combined Raman spectra data for the PLS model. 1st derivative and SNV preprocessing were applied to the combined Raman spectra data for the LASSO model (FIG. 10).

Chemometric models, as discussed herein, were built using the PLS model and the LASSO model. The PLS model and the LASSO model were modeled using MATLAB® version 9.14.0.2206163 (R2023a), as developed by The Math Works®, Inc., headquartered in Natick, Massachusetts.

Further discussion of the PLS model built using MATLAB® can be found on www.mathworks.com/help/stats/plsregress.html which is incorporated by reference herein in its entirety (last accessed 19 Jul. 2023). However, models built using various other programs and/or algorithms are envisioned within the scope of this disclosure.

The PLS model was built according to plsregress, which uses the SIMPLS algorithm. Plsregress provides for the function to center X and Y by subtracting the column means to provide a centered predictor and response variables X0 and Y0, respectively. This process does not rescale columns. To build the PLS model with standardized variables, zscore was used to normalize X and Y. Once X and Y were centered, plsregress was used to compute the singular value decomposition X0 and Y0. The PLS model will be discussed in greater below.

The PLS model was built where [XL, YL]=plsregress (X, Y,ncomp) which returns the predictor and response loadings XL and YL, respectively, for a partial least-squares (PLS) regression of the responses in matrix Y on the predictors in matrix X, using ncomp PLS components.

The PLS model was further built where [XL, YL,XS, YS, BETA, PCTVAR,MSE,stats] =pls regress (X, Y, ncomp) which returns predictor scores (XS); response scores (YS); a matrix (BETA) of coefficient estimates for PLS regression; a percentage of variance (PCTVAR) for the regression model; an estimated mean squares (MSE) for PLS models with ncomp components; and a structure stats that contain the PLS weights, T² statistic, with predictor and response residuals.

The PLS model was further built where [XL, YL,XS, YS,BETA, PCTVAR,MSE,stats] =plsregress (_,Name, Value) which provides options for using one or more name-value arguments in addition to any of the input argument combinations. The name-value arguments specify the MSE calculation parameters.

The pre-processed combined Spectra data was loaded onto MATLAB®. A predictor (x) was created representing a numeric matrix which contained the Raman spectra, and a response (y) was created representing a numeric vector which contained the corresponding concentration values. Perform PLS regression with LVs components (the number of components corresponding to the number of latent variable (LVs)) of the response in (y) on the predictors in (x), which is represented by [XL,yl,XS, YS,beta,PCTVAR]=plsregress (X,y,LVs).

The percent variance explained in the response variable (PCTVAR) was plotted as a function of the number of components. After the PCTVAR was plotted as a function of the number of components, compute a fitted response and display the residuals.

The input arguments are provided.

(1) provide the name-value arguments, such as, NameN=ValueN, where Name is the argument name and Value is the corresponding value. Name-Value arguments appear after other arguments, but no order is required for the pairs.

(2) provide cv-MSE calculation method. (1) Specify ‘cv’ as ‘resubstitution’ to use both X and Y to fit the model and estimate the mean squared errors, without cross-validation. (2) Specify ‘cv’ as a positive integer k to use k-fold cross-validation. (3) Specify ‘cv’ as a cvpartition object to specify another type of cross-validation partition.

(3) provide mcreps-Number of Monte Carlo repetitions. The Number of Monte Carlo repetitions for cross-validation, specified as a positive integer. Where, ‘cv’ was specified as ‘resubstitution,’ then mcreps was specified as 1.

The output arguments are provided.

(1) XL—Predictor loadings: XL is a p-by-ncomp matrix, where p is the number of predictor variables and ncomp is the number of PLS components.

(2) YL-Response loadings: YL is an m-by-ncomp matrix, where m is the number of response variables and ncomp is the number of PLS components.

(3) XS-Predictor scores: XS is an n-by-ncomp orthonormal matrix, where n is the number of observations and ncomp is the number of PLS components.

(4) YS-Response scores: YS is an n-by-ncomp matrix, where n is the number of observations and ncomp is the number of PLS components.

(5) BETA-Coefficient estimates for PLS regression: coefficient estimates for PLS regression are returned as a numeric matrix BETA is a (p+1)-by-m matrix, where p is the number of variables and m is the number of response variables.

(6) PCTVAR-Percentage of variance: percentage of variance explained by the model, is returned as a numeric matrix. PCTVAR is a 2-by-ncomp matrix, where ncomp is the number of PLS components.

(7) MSE-Means squared error: mean squared error is returned as a numeric matrix. MSE is a 2-by-(ncomp+1) matrix, where ncomp is the number of PLS components. MSE contains the estimated mean squared errors for a PLS model with ncomp components.

(8) stats-Model statistics: model statistics are returned as a structure, such as, W (p-by-ncomp matrix of PLS weight so that XS=Xθ*W), T2 (T² statistic for each point in XS), Xresiduals (Predictor residuals, Xθ-XS*XL′), Yresiduals (Response residuals, Yθ-XS*YL′).

Further discussion of the LASSO model built using MATLAB® can be found on www.mathworks.com/help/stats/lasso.html which is incorporated by reference herein in its entirety (last accessed 19 Jul. 2023). However, models built using various other programs and/or algorithms are envisioned within the scope of this disclosure. The above-indicated and possibly some other related problems in the state of the art can be beneficially addressed using various examples, aspects, features, and implementations of exemplary systems and methods for prediction-based Raman spectroscopy as disclosed herein. Accordingly, running one or more reactor for 6-15 runs per reactor is expensive and time consuming that the implementations described herein solve through particular computing systems and devices and computer-based prediction models. Thus, implementations disclosed herein provide improvements to chemometric-based Raman spectroscopy.

The PLS model was built according to LASSO, which used the Coordinate Descent algorithm.

Coordinate Descent algorithm provides using a covariance matrix to fit N data points and D predictors, where the fitting had a rough computational complexity of D*D. Without the use of the covariance matrix the computational complexity was about N*D. It was observed that using a covariance matrix was faster when N>D, and the default ‘auto’ setting of the UseCovariancce argument provided for the argument of N>D.

The PLS model was further built based on a matrix x of N p-dimensional normal variables. N is represented by a large variable and p=1000. Then a response a vector y is created from a model y=betaθ+X*p, where betaθ is a constant variable with added noise.

As described above in the detailed description, reference is made to the accompanying drawings that form a part hereof wherein like numerals designate like parts throughout, and in which is shown, by way of illustration, implementations that may be practiced. It is to be understood that other implementations may be utilized, and structured or logical changes may be made, without departing from the scope of the present disclosure. Therefore, the detailed description as described above is not to be taken in a limiting sense.

Various operations may be described as multiple discrete actions or operations in turn, in a manner that is most helpful in understanding the subject matter disclosed herein. However, the order of description should be construed as to imply that these operations are necessarily order dependent. In particular, these operations may not be performed in the order of presentation. Operations described may be performed in a different order from the described implementation. Various additional operations may be performed, and/or described operations may be omitted in additional implementations.

### CLAUSES

Implementations of the present disclosure are disclosed in the following clauses:
Clause 1. A computer-implemented method in an analytical instrument support apparatus, the method comprising:
   receiving, by one or more processors, a first spectra dataset associated with scans of one or more first samples, the one or more first samples including a target analyte having one or more known levels of a parameter;
   receiving, by one or more processors, a second spectra dataset associated with scans of one or more second samples;
   determining, by one or more processors, one or more spectra arrays by combining (i) the first spectra dataset and (ii) the second spectra dataset; and
   determining, by one or more processors, a chemometric model for one or more levels of the parameter of the target analyte based on, at least, the one or more spectra arrays and the one or more known levels of the parameter.
Clause 2. The computer-implemented method according to Clause 1, wherein the parameter is a concentration of the target analyte.
Clause 3. The computer-implemented method according to Clause 1 or Clause 2, the method further comprising:
   receiving, by one or more processors, a third spectra dataset from scans of a third sample, and determining, by one or more processors, one or more levels of the parameter of the target analyte in a third sample based on, at least, the determined chemometric model.
Clause 4. The computer-implemented method according to any one of Clauses 1-3, wherein the first spectra dataset and the second spectra dataset each include Raman shift wavenumbers.
Clause 5. The computer-implemented method according to any one of Clauses 1-4, wherein the target analyte is one or more of a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA, DNA, oligonucleotides, recombinant proteins, an edible product, polyols a polymer, or a molecule.
Clause 6. The computer-implemented method according to any one of Clauses 1-5, wherein the target analyte is glucose.
Clause 7. The computer-implemented method according to any one of Clauses 1-6, wherein the one or more second samples include a matrix.
Clause 8. The computer-implemented method according to any one of Clauses 1-7, wherein the matrix includes one or more of a serum, a protein, a nutrient, an intermediate of the target analyte, or a metabolite.
Clause 9. The computer-implemented method according to any one of Clauses 1-8, wherein the matrix includes a serum.
Clause 10. The computer-implemented method according to any one of Clauses 1-9, wherein the act of determining the one or more spectra arrays by combining includes summing the first spectra dataset and the second spectra dataset, the method further comprising:
   applying, by one or more processors, a weighted average to the summation of the first spectra dataset and the second spectra dataset, and generating, by one or more processors, the one or more spectra arrays based on, at least, the weighted average of the summation of the first spectra dataset and the second spectra dataset.
Clause 11. The computer-implemented method according to any one of Clause 1-10, wherein the determining of the one or more spectra arrays is based on, at least, combining (i) a proportional value between 0 and 1 from the first spectra dataset and (ii) a proportional value between 0 and 1 from the second spectra dataset between 0 and 1.
Clause 12. The computer-implemented method according to any one of Clauses 1-11, the method further comprising:
   applying, by one or more processors, one or more pre-processing operations to the first spectra dataset and the second spectra dataset, respectively, before the act of determining the one or more spectra arrays by combining (i) the first spectra dataset and (ii) the second spectra dataset, the pre-processing operations including region selection, spectra averaging, convolution filtering, 1st derivative, 2nd derivative, standard normal variate (SNV), multiplicative scatter correction, background removal, or any combinations thereof.
Clause 13. The computer-implemented method according to any one of Clauses 1-12, wherein the first spectra dataset is obtained from scans of the one or more first samples in one or more reactors, and the second spectra dataset is obtained from scans of the one or more second samples in one or more reactors.
Clause 14. The computer-implemented method according to any one of Clauses 1-13, wherein the one or more reactors have one or more physical properties including a volume, a form factor, one or more numbers and types of inlets and outlets, an interior surface area, one or more materials of construction, or any combinations thereof.
Clause 15. The computer-implemented method according to any one of Clauses 1-14, wherein the one or more reactors have one or more operational parameters including a feed type, a method of agitation, a pressure, a temperature, or any combinations thereof.
Clause 16. The computer-implemented method according to any one of Clauses 1-15, wherein the chemometric model is a partial least squares regression (PLS) model, principal component regression (PCR) model, least absolute shrinkage model and selection operator (LASSO) model, elastic-net regression model, support vector machine (SVM) model, or neural network model.
Clause 17. The computer-implemented method according to Clause 16, wherein the chemometric model is a PLS model or LASSO model.
Clause 18. A method for determining the one or more levels of the parameter of a target analyte based on the computer-implemented method according to claim 1, wherein the parameter is concentration.
Clause 19. One or more non-transitory computer-readable media having instructions stored thereon that, when executed by one or more processing devices of the analytical instrument support apparatus, cause the analytical instrument support apparatus to perform the computer-implemented method of Clause 1.
Clause 20. An analytical instrument support system comprising:
   one or more processors,
   one or more non-transitory computer-readable storage media; and
   program instructions stored on at least one of the one or more non-transitory computer-readable storage media for execution by at least one of the one or more processors, the program instructions comprising:
      program instructions to receive a first spectra dataset associated with scans of one or more first samples, the one or more first samples including a target analyte having one or more known levels of a parameter;
      program instructions to receive a second spectra dataset associated with scans of one or more second samples;
      program instructions to determine one or more spectra arrays by combining (i) the first spectra dataset and (ii) the second spectra dataset; and
      program instructions to determine a chemometric model for one or more levels of the parameter of the target analyte based on, at least, the one or more spectra arrays and the one or more known levels of the parameter.
Clause 21. The analytical instrument support system according to Clause 20, wherein the program instructions are executed on a common computing device including at least one of the one or more processors.
Clause 22. The analytical instrument support system according to any one of Clauses 20 or 21, wherein the program instructions are executed on a computing device including at least one of the one or more processors, and wherein the computing device is remote from an analytical instrument associated with the analytical instrument support system.
Clause 23. The analytical instrument support system according to any one of Clauses 20 to 22, wherein the program instructions are executed on a user computing device including at least one of the one or more processors.
Clause 24. The analytical instrument support system according to any one of Clauses 20 to 23, wherein at least one of the one or more processors is disposed in an analytical instrument associated with the analytical instrument support system, and wherein the program instructions are executed on the at least one of the one or more processors.
Clause 25. An analytical instrument comprising:
   a light source configured to direct light onto a surface of a sample;
   a spectrograph configured to acquire a Raman spectrum from the surface of the sample in response to the light source directing light onto the surface of the sample;
   one or more processors;
   one or more non-transitory computer-readable storage media; and
   program instructions stored on at least one of the one or more non-transitory computer-readable storage media for execution by at least one of the one or more processors, wherein execution of the program instructions by at least one of the one or more processors, cause the analytical instrument to implement the following acts, comprising:
      receiving a first spectra dataset associated with scans of one or more first samples, the one or more first samples including a target analyte having one or more known levels of a parameter;
      receiving a second spectra dataset associated with scans of one or more second samples;
      determining one or more spectra arrays by combining (i) the first spectra dataset and (ii) the second spectra dataset; and
      determining a chemometric model for one or more levels of the parameter of the target analyte based on, at least, the one or more determined spectra arrays and the one or more known levels of the parameter.
Clause 26. The analytical instrument according to Clause 25, the analytical instrument further comprising:
   determining one or more levels of the parameter of the target analyte in a third sample based on, at least, a third spectral dataset from a scan of the third sample and the determined chemometric model.
Clause 27. The analytical instrument according to either of Clauses 25 or 26, wherein the target analyte is one or more of one or more of a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA, DNA, oligonucleotides, recombinant proteins, an edible product, polyols a polymer, or a molecule.
Clause 28. The analytical instrument according to any of Clauses 25-27, wherein the target analyte is glucose.
Clause 29. The analytical instrument according to any of Clauses 25-28, wherein the one or more second samples include a matrix.
Clause 30. The analytical instrument according to Clause 29, wherein the matrix includes one or more of a serum, a protein, a nutrient, an intermediate of the target analyte, or a metabolite.
Clause 31. The analytical instrument according to Clause 30, wherein the matrix includes a serum.
Clause 32. The analytical instrument according to any of Clauses 25-31, wherein the act of determining the one or more spectra arrays by combining includes summing the first spectra dataset and the second spectra dataset, wherein execution of the program instructions by at least one of the one or more processors cause the analytical instrument to implement further acts, comprising:
   applying a weighted average to the summation of the first spectra dataset and the second spectra dataset; and
   generating the one or more spectra arrays based on, at least, the weighted average of the summation of the first spectra dataset and the second spectra dataset.
Clause 33. The analytical instrument according to any one of Clause 25-32, wherein the first spectra dataset is obtained from scans of the one or more first samples in one or more reactors, and the second spectra dataset is obtained from scans of the one or more second samples in one or more reactors.
Clause 34. The analytical instrument according to Clause 33, wherein the first spectra dataset and the second spectra dataset are associated with one or more reactors, wherein the one or more reactors have one or more physical properties including a volume, a form factor, one or more numbers and types of inlets and outlets, an interior surface area, one or more materials of construction, or any combinations thereof.
Clause 35. The analytical instrument according to Clause 33, wherein the one or more reactors have one or more operational parameters including a feed type, a method of agitation, a pressure, a temperature, or any combinations thereof.
Clause 36. The analytical instrument according to any of Clause 25-35, wherein the chemometric model is a partial least squares regression (PLS) model, a principal component regression (PCR) model, least absolute shrinkage and selection operator (LASSO) model, an elastic net regression model, a support vector machine (SVM) model, or a neural network.
Clause 37. The analytical instrument according to Clause 36, wherein the chemometric model is a PLS model or a LASSO model.
Clause 38. The analytical instrument according to any of Clauses 25-37, the analytical instrument further comprising:
   applying one or more pre-processing operations to the first spectra dataset and the second spectra dataset, respectively, before the act of generating the one or more spectra arrays by combining (i) the first spectra dataset and (ii) the second spectra dataset, the one or more pre-processing operations including region selection, spectra averaging, convolution filtering, 1^{st} derivative, 2^{nd} derivative, standard normal variate, multiplicative scatter correction, background removal, or any combinations thereof.

What is claimed is:

1. A computer-implemented method on an analytical instrument support apparatus, the method comprising:
receiving, by one or more processors, a first spectra dataset associated with first scans of one or more first samples, the one or more first samples including a target analyte having one or more known levels of a parameter;
receiving, by the one or more processors, a second spectra dataset associated with second scans of one or more second samples;
determining, by the one or more processors, one or more spectra arrays by combining (i) the first spectra dataset and (ii) the second spectra dataset; and
determining, by the one or more processors, a chemometric model for one or more levels of the parameter of the target analyte based on, at least, the one or more spectra arrays and the one or more known levels of the parameter.

2. The computer-implemented method according to claim 1, wherein the parameter is a concentration of the target analyte.

3. The computer-implemented method according to claim 1, the method further comprising:
receiving, by the one or more processors, a third spectra dataset from third scans of a third sample, and determining, by one or more processors, one or more levels of the parameter of the target analyte in a third sample based on, at least, the chemometric model.

4. The computer-implemented method according to claim 1, wherein the first spectra dataset and the second spectra dataset each include Raman shift wavenumbers.

5. The computer-implemented method according to claim 1, wherein the target analyte is one or more of a metabolite, an antigen, an antibody, a viral vector, a vaccine, a bacteria, a yeast, a fungus, a toxin, pharmaceutical drugs, steroids, lipids, a protein, a vitamin, an enzyme, blood, blood components, cells, allergens, tissues, RNA, DNA, oligonucleotides, recombinant proteins, an edible product, polyols a polymer, or a molecule.

6. The computer-implemented method according to claim 1, wherein the target analyte is glucose.

7. The computer-implemented method according to claim 1, wherein the one or more second samples include a matrix.

8. The computer-implemented method according to claim 7, wherein the matrix includes one or more of a serum, a protein, a nutrient, an intermediate of the target analyte, or a metabolite.

9. The computer-implemented method according to claim 8, wherein the matrix includes a serum.

10. The computer-implemented method according to claim 1, wherein determining the one or more spectra arrays includes determining a summation of the first spectra dataset and the second spectra dataset, the method further comprising:
applying, by the one or more processors, a weighted average to the summation of the first spectra dataset and the second spectra dataset, and
generating, by the one or more processors, the one or more spectra arrays based on, at least, the weighted average of the summation of the first spectra dataset and the second spectra dataset.

11. The computer-implemented method according to claim 1, wherein determining the one or more spectra arrays includes combining (i) a proportional value between 0 and 1 from the first spectra dataset and (ii) a proportional value between 0 and 1 from the second spectra dataset.

12. The computer-implemented method according to claim 10, the method further comprising:
applying, by the one or more processors, one or more pre-processing operations to the first spectra dataset and the second spectra dataset, respectively, before determining the one or more spectra arrays by combining (i) the first spectra dataset and (ii) the second spectra dataset, the one or more pre-processing operations including at least one selected from a group consisting of region selection, spectra averaging, convolution filtering, 1st derivative, 2nd derivative, standard normal variate (SNV), multiplicative scatter correction, and background removal.

13. The computer-implemented method according to claim 1, wherein the first spectra dataset is obtained from the first scans of the one or more first samples in one or more reactors, and the second spectra dataset is obtained from the second scans of the one or more second samples in the one or more reactors.

14. The computer-implemented method according to claim 13, wherein the one or more reactors have one or more physical properties including a volume, a form factor, one or more numbers and types of inlets and outlets, an interior surface area, one or more materials of construction, or any combinations thereof.

15. The computer-implemented method according to claim 13, wherein the one or more reactors have one or more operational parameters including a feed type, a method of agitation, a pressure, a temperature, or any combinations thereof.

16. The computer-implemented method according to claim 1, wherein the chemometric model is one selected from a group consisting of a partial least squares regression (PLS) model, a principal component regression (PCR) model, a least absolute shrinkage model and selection operator (LASSO) model, an elastic-net regression model, a support vector machine (SVM) model, and a neural network model.

17. The computer-implemented method according to claim 16, wherein the chemometric model is a PLS model or LASSO model.

18. An analytical instrument support system comprising:
one or more processors,
one or more non-transitory computer-readable storage media; and
program instructions stored on at least one of the one or more non-transitory computer-readable storage media for execution by at least one of the one or more processors to perform a set of functions, the set of functions comprising:
receiving a first spectra dataset associated with first scans of one or more first samples, the one or more first samples including a target analyte having one or more known levels of a parameter;
receiving a second spectra dataset associated with second scans of one or more second samples;
determining one or more spectra arrays by combining (i) the first spectra dataset and (ii) the second spectra dataset; and
determining a chemometric model for one or more levels of the parameter of the target analyte based on, at least, the one or more spectra arrays and the one or more known levels of the parameter.

19. An analytical instrument comprising:
a light source configured to direct light onto a surface of a sample;
a spectrograph configured to acquire a Raman spectrum from the surface of the sample in response to the light source directing light onto the surface of the sample;
one or more processors;
one or more non-transitory computer-readable storage media; and
program instructions stored on at least one of the one or more non-transitory computer-readable storage media for execution by at least one of the one or more processors, wherein execution of the program instructions by at least one of the one or more processors cause the analytical instrument to perform a set of functions, the set of functions comprising:
receiving a first spectra dataset associated with first scans of one or more first samples, the one or more first samples including a target analyte having one or more known levels of a parameter;
receiving a second spectra dataset associated with second scans of one or more second samples;
determining one or more spectra arrays by combining (i) the first spectra dataset and (ii) the second spectra dataset; and
determining a chemometric model for one or more levels of the parameter of the target analyte based on, at least, the one or more spectra arrays and the one or more known levels of the parameter.

20. The analytical instrument according to claim 19, wherein determining the one or more spectra arrays includes:
determining a summation of the first spectra dataset and the second spectra dataset;
applying a weighted average to the summation of the first spectra dataset and the second spectra dataset; and
generating the one or more spectra arrays based on, at least, the weighted average of the summation of the first spectra dataset and the second spectra dataset.
